(19) United States
(12) Patent Application Publication
(10) Pub. No.: US 2011/0250284 A1
(43) Pub. Date: Oct. 13, 2011

(54) Nanoparticles for Use as Synthetic Platelets and Therapeutic Agent Delivery Vehicles

(76) Inventors: Erin B. Lavik, Cleveland Heights, OH (US); James P. Bertram, New Haven, CT (US); Stephany Y. Tzeng, Balitmore, MD (US)

(21) Appl. No.: 13/001,283

(22) PCT Filed: Jun. 22, 2009
(86) PCT No.: PCT/US2009/048141

§ 371 Date: Jul. 1, 2011

Related U.S. Application Data
(60) Provisional application No. 61/075,085, filed on Jun. 24, 2008.

(51) Int. Cl. A61K 9/50; A61K 38/07; A61K 47/48; A61K 38/18; A61P 7/04; A61P 25/00; A61K 38/06; A61K 38/08

(52) U.S. Cl. 424/497; 424/78.17; 514/7.6; 525/54.1

(57) ABSTRACT

The invention relates to synthetic platelet compositions and methods useful in diminishing bleeding and blood loss. The synthetic platelets of the invention can comprise a biocompatible, biodegradable polymer, including, for example, a poly(lactic-co-glycolic acid)-poly-L-lysine (PLGA-PLL) block copolymer, having conjugated PEG arms terminating with RGD motif containing peptides. The invention further comprises compositions and methods useful in the delivery of therapeutic agents.

### BACKGROUND OF THE INVENTION

Traumatic injury is the leading cause of death for individuals between the ages of 5 and 44 (Krug et al., 2000, American Journal of Public Health 90, 523-526), and blood loss is the major factor in both civilian and battlefield traumas (Champion et al., 2003, Journal of Trauma-Injury Infection and Critical Care 54, S13-S19; Sauaia et al., 1995, Journal of Trauma-Injury Infection and Critical Care 38, 185-193). Following injury, hemostasis is established through a series of coagulatory events including platelet activation. However, with severe injuries, these processes are insufficient and result in uncontrolled bleeding. Methods to staunch bleeding have included pressure dressings and absorbent materials (e.g. Quik-clot®), but these treatments are limited to compressible and exposed wounds. Alternatives have included allogeneic platelet transfusions, clotting factors, and platelet substitutes, but efficacy, immunogenicity, and thrombosis have stalled their application (Kim & Greenburg, 2006, Artificial Cells Blood Substitutes and Biotechnology 34, 537-550). Immediate intervention is one of the most effective means of minimizing mortality associated with severe trauma (Regel & Seekamp, 1997, Acta Anaesthesiol Scand Suppl 110, 71-76).

Administration of allogenic platelets are a logical means to halt bleeding; however, platelets have a short shelf life, and administration of allogenic platelets can cause graft versus host disease, alloimmunization, and transfusion-associated lung injuries (Blajchman, 1999, Nature Medicine 5, 17-18). Recombinant factors including Factor VIIa (NovoSeven®) can augment hemostasis, but immunogenic and thromboembolic complications are unavoidable risks (Benharash & Putnam, 2005, (Southeastern Surgical Congress, Santa Barbara, Calif.) 776-780; Boffard et al., 2004, In 63rd Annual Meeting of the American-Association-for-the-Surgery-of-Trauma 8-16 (Maui, Hi.)). Nonetheless, NovoSeven® has become the standard of care in a number of trauma and surgical situations where bleeding cannot otherwise be controlled (Benharash & Putnam, 2005, (Southeastern Surgical Congress, Santa Barbara, Calif.) 776-780). Non-platelet alternatives including red blood cells modified with the Arg-Gly-Asp (RGD) sequence, fibrinogen-coated microcapsules based on albumin, and liposomal systems have been studied as coagulants (Lee & Blajchman, 2001, British Journal of Haematology 114, 496-505), but toxicity, thrombosis, and limited efficacy have stalled many of these products (Kim & Greenburg, 2006, Artificial Cells Blood Substitutes and Biotechnology 34, 537-550).

In the past, composites of microspheres for therapeutic agent delivery with a tissue engineering scaffold that provides a substrate for cell growth have been studied (Nkansah et al., 2008, Biotech Bioeng 100:1010-9), most of these created by the suspension of the therapeutic agent delivery microspheres within the hydrogel; the possibility of using covalent bonds between the particles and the hydrogel to strengthen the system has not been investigated.

Hydrogels have been found to be a particularly good material for tissue engineering scaffolds for several reasons. Their high water content mimics soft tissue content—the mechanical properties provide a suitable environment for cell culture because of closer similarity to tissues in the body and because the gel is less likely to cause further trauma or irritation in contrast to the relatively hard scaffolds made by more traditional methods with polymers like Poly(lactic-co-glycolic acid) (PLGA) (Hong et al., 2007, J Biomed Mat Res 85A:628-37). In particular, photopolymerizable hydrogels present a promising possibility because they can be injected locally, cured very quickly, and used to deliver other small particles into the body along with the gel macromer solution (Brandl et al., 2006, Biomaterials 28:134-46).

The growth factor ciliary neurotrophic factor (CNTF) has been shown to have a protective effect on motor neurons following injury to the adult central nervous system (CNS) (Clatterbuck, 1993, Proc Natl Acad Sci USA 90:2222-2226) and on neurons and photoreceptors in degenerative diseases (Clatterbuck, 1993, Proc Natl Acad Sci USA 90:2222-2226; Emerich, 1997, Nature 386:395-9). CNTF may also have a role in directing neural stem cells (NSCs) to differentiate into mature cells (Sendtner, 1992, Nature 358:502-4). Combined with its neuroprotective abilities, the potential effects of CNTF on differentiation of progenitor cells suggest that it may be valuable in treatments for trauma to the CNS or to degenerative diseases.

However, because CNTF causes numerous side effects at high levels (Bonni, 1997, Science 278:477-83), sustained, local delivery of CNTF is crucial for its application. PLGA microspheres and nanoparticles may be useful to control the delivery of CNTF, as well as to direct the differentiation of NSCs to mature cell fates (Amyotrophic lateral sclerosis (ALS) CNTF Treatment Study Group, 1996, Neurology 46:1244-9), but PLGA on its own does not lend itself to targeted administration within the body.

Therefore, there exists a need in the art for safe compositions and methods useful for diminishing bleeding. Moreover, there remains a need in the art for compositions and methods useful for the prolonged delivery of therapeutic agents, such as CNTF, that are useful in therapeutic agent therapies, such as, for example, the treatment of trauma or degenerative disease involving the nervous system.

### BRIEF SUMMARY OF THE INVENTION

The present invention comprises synthetic platelet compositions and methods useful in diminishing bleeding and blood loss. The invention further comprises nanoparticle therapeutic agent delivery vehicle compositions and methods useful in the delivery of therapeutic agents.

The synthetic platelet compositions generally comprise a biocompatible, biodegradable polymer, such as a polyhydroxy acid polymer, conjugated with at least one polyethylene glycol molecule, which has been conjugated with at least one RGD motif containing peptide. In some embodiments, the polymer comprises at least one of poly-lactic-co-glycolic acid and poly-L-lactic acid. In certain embodiments, the polymer comprises a poly(lactic-co-glycolic acid)-poly-L-lysine (PLGA-PLL) copolymer. The polyethylene glycol molecule may be at least one of PEG 200, PEG 1000, PEG 1500, PEG 4600 and PEG 10,000. RGD motif containing peptides useful in the invention include, Arg-Gly-Asp (RGD) (SEQ ID NO: 1), Arg-Gly-Asp-Ser (RGDS) (SEQ ID NO: 2), and Gly-Arg-Gly-Asp-Ser (GRGDS) (SEQ ID NO: 3). In certain embodiments, the synthetic platelet composition further comprises a pharmaceutically acceptable carrier. In various embodiments, the invention includes methods of using the synthetic platelet compositions of the invention to diminishing bleeding in a subject in need thereof, the methods comprising administering to the subject a therapeutically effective amount of the synthetic platelet compositions described herein.

The nanoparticle therapeutic agent delivery vehicle compositions generally comprise a biocompatible, biodegradable polymer, such as a polyhydroxy acid polymer, conjugated with at least one polyethylene glycol acrylate molecule, the nanoparticle encapsulating at least one therapeutic agent. In some embodiments, the polymer comprises at least one of poly-lactic-co-glycolic acid and poly-L-lactic acid. In certain embodiments, the polymer comprises a poly(lactic-co-glycolic acid)-poly-L-lysine (PLGA-PLL) copolymer. The polyethylene glycol acrylate molecule may be at least one of PEG 200, PEG 1000, PEG 1500, PEG 4600 and PEG 10,000. In some embodiments, the therapeutic agent is CNTF. In certain embodiments, the nanoparticle therapeutic agent delivery vehicle composition further comprises a pharmaceutically acceptable carrier. In various embodiments, the invention includes methods of nanoparticle therapeutic agent delivery compositions of the invention to treat a disorder in a subject in need thereof, the methods comprising administering to the subject a therapeutically effective amount of the nanoparticle therapeutic agent delivery vehicle compositions described herein.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts schematic of a synthetic platelet and a scanning electron micrograph. (a) Schematic of synthetic platelet comprised of Poly(lactic-co-glycolic acid)(PLGA)-Poly(ε-carbobenzoxy-L-lysine)(PLL) (PLGA-PLL) core with polyethylene glycol (PEG) arms terminated with the RGD moiety. (b) Scanning electron microscope (SEM) micrograph of synthetic platelets. Scale bar=1 μm. (c) Lysine and peptide concentrations of synthetic platelets as determined by amino acid (AA) analysis. Conjugation efficiency was defined as the peptide to lysine ratio multiplied by 100. (d) Diameter of PLGA-PLL core and PLGA-PLL-PEG nanoparticles as determined by SEM microscopy and dynamic light scattering (DLS). SEM diameter based on n=80. Data are expressed as mean±SD.

FIG. 2 depicts a schematic of the in vitro characterization of polymers' interactions with activated platelets. (a) Schematic of in vitro assay for quantifying platelet adhesion to polymers. 5-chloromethylfluorescein diacetate (CMFDA) labeled platelets' adhesion to polymers following agitation and addition of ADP; (b) PEG 4600 and (c) 4600-GRGDS. Scale bar=500 (d) Quantification of platelet adhesion. Area of fluorescence represents area of platelet aggregation (n=3). Data are expressed as mean±SE (*P<0.05).

FIG. 3 depicts the results of an example experiment conducting in vivo analysis of bleed time and biodistribution of synthetic platelets. (a) Bleed times in femoral artery injury following intravenous administration of the synthetic platelets (n=5). Data presented as % of ‘No injection’ mean. No Injection=240±15 seconds. Data are expressed as mean±SE (*P<0.05 and ***P<0.001 versus saline, and #P<0.05 versus rFVIIa). (b) Injured femoral artery and blood spurting from injury. Arrow demarcates injury site. (c) SEM micrograph of clot excised from injured artery following synthetic platelet administration (4600-GRGDS). Synthetic platelets intimately associated with clot and connecting fibrin mesh (arrow). Scale bar=1 μm. (d) Cross-section of clot following femoral artery injury and injection of C6 labeled synthetic platelets. Blue is DAPI labeled nuclei of smooth muscle and endothelial cells and green is C6 from synthetic platelets within clot. Scale bar=100 μm. (e) Biodistribution of 4600-GRGDS synthetic platelets. No fluorescence was detected at 3 and 7 day time points following injection (n=3). Data are expressed as mean±SE. (f) HPLC quantification of clot associated C6 following injury (n=5). Data expressed as mean±SE (**P<0.01).

FIG. 4 depicts polymer synthesis and characterization. (a) Reaction scheme for PLGA-PLL-PEG-RGD polymer. (b) Conjugation/deprotection was verified using ultraviolet-visible spectroscopy (UV-vis). (c) 1H NMR was utilized for determining conjugation of PEG to PLGA-PLL. (d) The successful conjugation of RGD was partially determined using attenuated total reflectance Fourier transform infrared spectroscopy (ATR-FTIR).

FIG. 5 depicts polymer characterization and in vitro assay with collagen. (a) Lysine and peptide concentrations of PLGA-PLL-PEG-RGD polymer as determined by AA analysis. Conjugation efficiency was defined as the peptide to lysine ratio multiplied by 100. PLGA-PLL-PEG-RGD polymer was used for in vitro studies. Polymer was fabricated as described in FIG. 4a. (b) Quantification of CMFDA-labeled platelet aggregation in wells coated with Collagen I (rat tail). Comparison between wells with and without ADP. Experiments were performed in triplicate. Data expressed as mean±SE (**P<0.01).

FIG. 6 depicts the results of an example experiment evaluating in vivo hemostatic properties of synthetic platelets. (a) Bleed times following the administration of PEG 1500 synthetic platelets (n=5). Data presented as % of ‘No injection’ mean. No Injection bleed time was 240±15 seconds. Data are expressed as mean±SE (**P<0.01 and ***P<0.001). (b) SEM micrograph of clot excised from injured artery following synthetic platelet administration (4600-GRGDS). Arrow demarcates synthetic platelets in clot. Clot imaged from lumen side. Scale bar=2 μm.

FIG. 7 depicts the results of an example experiment assessing the biodistribution of 4600-GRGDS synthetic platelets following femoral artery injury. (a) In vitro evaluation of cumulative C6 released from C6 loaded synthetic platelets over 7 days. Data presented as mean±SD (n=3). (b) Biodistribution of 4600-GRGDS synthetic platelets immediately following femoral artery injury (n=3). Because synthetic platelets were allowed to circulate for 5 minutes prior to injury, and the injury bleeds for approximately 3 minutes, this time is compared to 10 minute biodistribution with no injury. Data presented as mean±SE (c) Biodistribution of 4600-GRGDS synthetic platelets 1 hour following femoral artery injury (n=3). Data presented as mean±SE.

FIG. 8 depicts the results of an example experiment conducting in vivo analysis of bleed times in femoral artery injury following post-injury intravenous administration of the synthetic platelets.

FIG. 9 depicts a reaction scheme for PLGA-b-PLL-g-PEG acrylate.

FIG. 10 depicts 1H-NMR spectra. Circled peaks represent the protons of the acrylate group (5.87, 6.17, 6.42 ppm). A: PEG acrylate (top) and PEG (bottom). B: PLGA-b-PLL-g-(PEG acrylate) (top) and PLGA (bottom).

FIG. 11 depicts the results of an example experiment assessing CNTF release profiles from PLGA nanoparticles, copolymer nanoparticles, and copolymer nanoparticles encapsulated in hydrogel.

FIG. 12 depicts the results of an example experiment demonstrating that the elastic modulus of gels decreases when nanoparticles are added to the hydrogel, but the presence of acrylate groups on the nanoparticles partially compensates by forming additional crosslinks.

FIG. 13 depicts the results of an example experiment assessing physiologic responses to CNTF. FIG. 13A: Migration from neurospheres. FIG. 13B: Expression.

FIG. 14 depicts the results of an example experiment assessing NSC's encapsulated in PEG hydrogels. NSCs encapsulated in PEG hydrogels (A) show little migration, in contrast to those encapsulated in hydrogels with both PEG and PLL (B).

FIG. 15 depicts the results of an example experiment assessing NSC's encapsulated in PEG hydrogels. In each pair of images, the left shows nestin (A-B, E-F) or GFAP (C-D, G-H) expression and the right shows expression of the protein, cell bodies (GFP), and nuclei (DAPI).

FIG. 16 depicts the results of an example experiment evaluating the stability of the synthetic platelets at room temperature.

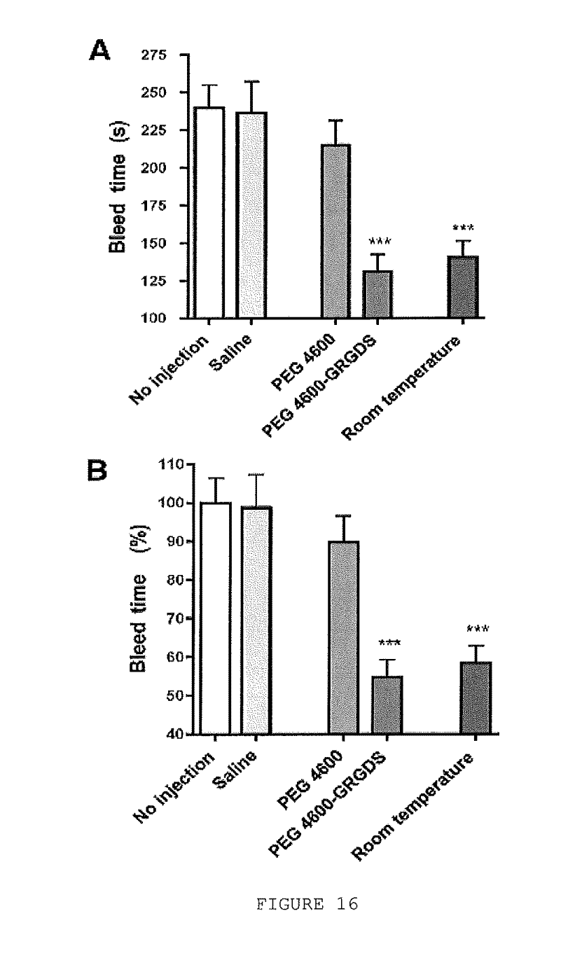

### DETAILED DESCRIPTION

The present invention comprises compositions and methods useful in diminishing bleeding and blood loss. The invention further comprises compositions and methods useful in the delivery of therapeutic agents.

### Definitions:

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The terms “activate” or “activation” as used herein with reference to a biologically active molecule or biochemical pathway, such as a clotting cascade, indicates any modification in the genome and/or proteome of an organism that increases the biological activity of the biologically active molecule or biochemical pathway in the organism. Exemplary activations include but are not limited to modifications that results in the conversion of the molecule from a biologically inactive form to a biologically active form and from a biologically active form to a biologically more active form, and modifications that result in the expression of the biologically active molecule or biochemical pathway in an organism wherein the biologically active molecule or biochemical pathway was previously not expressed. For example, activation of a biologically active molecule or biochemical pathway can be performed by expressing a native or heterologous polynucleotide encoding for the biologically active molecule or biochemical pathway in the organism, by expressing a native or heterologous polynucleotide encoding for an enzyme involved in the pathway for the synthesis of the biological active molecule in the organism, by expressing a native or heterologous molecule that enhances the expression of the biologically active molecule or biochemical pathway in the organism.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “biochemical pathway” refers to a connected series of biochemical reactions normally occurring in a cell or in an organism such as, for example, a clotting cascade. Typically, the steps in such a biochemical pathway act in a coordinated fashion to produce a specific product or products or to produce some other particular biochemical or physiologic action.

A “conservative substitution” is the substitution of an amino acid with another amino acid with similar physical and chemical properties. In contrast, a “nonconservative substitution” is the substitution of an amino acid with another amino acid with dissimilar physical and chemical properties.

As used herein, “homology” is used synonymously with “identity.”

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

The terms “inactivate” or “inactivation” as used herein with reference to a biologically active molecule or biochemical pathway, indicates any modification in the genome and/or proteome of an organism that prevents or reduces the biological activity of the biologically active molecule or biochemical pathway in the organism. Exemplary inactivations include but are not limited to modifications that results in the conversion of the molecule from a biologically active form to a biologically inactive form and from a biologically active form to a biologically less or reduced active form, and any modifications that result in a total or partial deletion of the biologically active molecule. For example, inactivation of a biologically active molecule or biochemical pathway can be performed by deleting or mutating the a native or heterologous polynucleotide encoding for the biologically active molecule or biochemical pathway in the organism, by deleting or mutating a native or heterologous polynucleotide encoding for an enzyme involved in the pathway for the synthesis of the biologically active molecule or biochemical pathway in the organism, by activating a further a native or heterologous molecule that inhibits the expression of the biologically active molecule or biochemical pathway in the organism.

The term “modulate,” as used herein, refers to any change from the present state. The change may be an increase or a decrease. For example, the activity of a biologically active molecule or biochemical pathway may be modulated such that the activity of the biologically active molecule or biochemical pathway is increased from its current state. Alternatively, the activity of an enzyme may be biologically active molecule or biochemical pathway such that the activity of the biologically active molecule or biochemical pathway is decreased from its current state.

The terms “diminishing,” “reducing,” or “preventing,” “inhibiting,” and variations of these terms, as used herein include any measurable decrease, including complete or substantially complete inhibition.

The term “nucleic acid” typically refers to a large polynucleotide.

A “polynucleotide” means a single strand or parallel and anti-parallel strands of a nucleic acid. Thus, a polynucleotide may be either a single-stranded or a double-stranded nucleic acid.

The term “oligonucleotide” typically refers to short a polynucleotide, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, enhancer, origin of replication, ribosome-binding site, etc.) as well.

A “recombinant polypeptide” is one which is produced upon expression of a recombinant polynucleotide.

“Mutants,” “derivatives,” and “variants” of a polypeptide (or of the DNA encoding the same) are polypeptides which may be modified or altered in one or more amino acids (or in one or more nucleotides) such that the peptide (or the nucleic acid) is not identical to the wild-type sequence, but has homology to the wild type polypeptide (or the nucleic acid).

A “mutation” of a polypeptide (or of the DNA encoding the same) is a modification or alteration of one or more amino acids (or in one or more nucleotides) such that the peptide (or nucleic acid) is not identical to the sequences recited herein, but has homology to the wild type polypeptide (or the nucleic acid).

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “portion” of a polypeptide means at least about three sequential amino acid residues of the polypeptide. It is understood that a portion of a polypeptide may include every amino acid residue of the polypeptide.

The term “therapeutic agent,” as used herein, is defined as a substance capable of administration to an animal, preferably a human, which modulates the animals physiology. More preferably the term “therapeutic agent,” as used herein, is defined as any substance intended for use in the treatment or prevention of disease in an animal, preferably in a human. Therapeutic agent includes synthetic and naturally occurring bioaffecting substances, as well as recognized pharmaceuticals, such as those listed in “The Physicians Desk Reference,” 61st edition (2007), “Goodman and Gilman's The Pharmacological Basis of Therapeutics” 10th Edition (2001), and “The United States Pharmacopeia, The National Formulary”, USP XXX NF XXV (2007), the compounds of these references being herein incorporated by reference. The term therapeutic agent also includes compounds that have the indicated properties that are not yet discovered. The term therapeutic agent includes pro-active, activated and metabolized forms of therapeutic agents.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

### Description

### Synthetic Platelets

In various embodiments, the present invention provides synthetic platelets having Arg-Gly-Asp (RGD) functionalized nanoparticles and methods of their use.

It is an aspect of the invention that administration, such as for example intravenous administration, of the synthetic platelets of the invention can diminish the bleeding time in an subject. It is a further aspect of the invention that the synthetic platelets provide a nanostructure that binds with activated platelets and enhances their rate of aggregation to aid in stopping bleeding.

The synthetic platelets of the present invention can comprise a biocompatible, biodegradable polymer, including, for example, polyhydroxy acid polymers, such as poly-lactic-co-glycolic acid and poly-L-lactic acid, with conjugated PEG arms terminating with RGD motif containing peptides. In preferred embodiments, the synthetic platelets comprise poly(lactic-co-glycolic acid)-poly-L-lysine (PLGA-PLL) block copolymer cores with conjugated PEG arms terminating with RGD motif containing peptides.

### Nanoparticles

In various embodiments, compositions of the invention comprise a nanoparticle. In some embodiments, the nanoparticles of the invention comprise a synthetic platelet. In other embodiments, the nanoparticles of the invention comprise a therapeutic agent delivery vehicle.

In various embodiments, the nanoparticles of the present invention comprise a biocompatible, biodegradable polymer, including, for example, polyhydroxy acid polymers such as poly-lactic-co-glycolic acid (PLGA) and poly-lactic acid (PLA), or combinations thereof In other embodiments, the nanoparticles of the present invention comprise a biocompatible, biodegradable polymer, including, for example, poly-lactic-co-glycolic acid (PLGA), poly-lactic acid (PLA), polyethylene glycol (PEG) or combinations thereof In certain embodiments, the nanoparticles of the invention comprise poly(lactic-co-glycolic acid)-poly-L-lysine (PLGA-PLL). In various embodiments, a metal (such as, for example, gold), or a ceramic, or a polystyrene core, onto which PEG is conjugated, may be used.

In various embodiments, the nanoparticles of the present invention are modified by conjugating them with PEG molecules of a variety of molecular weights, including, for example, PEG 200, PEG 1000, PEG 1500, PEG 4600, PEG 10,000, or combinations thereof. In other embodiments, the nanoparticles of the present invention are modified by conjugating them with PEG acrylate, or PEG diacrylate, molecules of a variety of molecular weights.

In some embodiments, the nanoparticles of the present invention are also modified by conjugating them with an RGD motif containing peptide, such as, for example, Arg-Gly-Asp (RGD) (SEQ ID NO: 1), Arg-Gly-Asp-Ser (RGDS) (SEQ ID NO: 2). Gly-Arg-Gly-Asp-Ser (GRGDS) (SEQ ID NO: 3) or a control peptide, such as, for example, Gly-Arg-Ala-Asp-Ser-Pro (GRADSP) (SEQ ID NO: 4)). The RGD motif containing peptide of the invention may contain a single repeat of the RGD motif or may contain multiple repeats of the RGD motif, such as, for example, 2, or 5, or 10 or more repeats of the RGD motif. One of skill in the art will understand that conservative substitutions of particular amino acid residues of the RGD motif containing peptides of the invention may be used so long as the RGD motif containing peptide retains the ability to bind comparably as the native RGD motif. One of skill in the art will also understand that conservative substitutions of particular amino acid residues flanking the RGD motif so long as the RGD motif containing peptide retains the ability to bind comparably as the native RGD motif.

In still further embodiments, the nanoparticles of the present invention are modified by conjugating them with a peptide having a motif other than, or in addition to, an RDG motif. By way of a non-limiting example, KQAGDV (SEQ ID NO: 5), which is present in the carboxy-terminus of the fibrinogen gamma-chain (Kloczewiak et al., 1982, Biochemical and Biophysical Research Communications 107: p. 181-187) and which appears to mimic the RGD sequence binding to receptor GPIIb-IIIa, may be used (Lam et al., 1987, Journal of Biological Chemistry 262:947-950). By way of another non-limiting example, KGD (SEQ ID NO: 6), which has also been shown to bind to GP IIb-IIIa, may be used (Plow et al., 1985, P.N.A.S. 82:8057-8061. By way of further non-limiting examples, polypeptides such as fibrinogen, fibronectin, vitronectin, vonWillebrand factor, or fragments or combinations thereof, may be used (Pytela et al., 1986, Science 231:1559-1562; Gardner, 1985, Cell 42:439-448). One of skill in the art will understand that many peptides and polypeptides can be conjugated to the synthetic platelet of the invention, so long as the peptide or polypeptide is able to bind with activated platelets.

In some embodiments, the nanoparticles range in diameter from about 1 nM to about 500 nM in diameter. In other embodiments, the nanoparticles range in diameter from about 1 nM to about 1 μM.

### Methods of Making a Synthetic Platelet

The polymer-based nanoparticle synthetic platelets of the present invention may be prepared in accordance with the following method.

A copolymer, such as, for example, PLGA-PLL is synthesized as follows. Briefly, each of the polymer materials, such as PLGA 503H and Poly(ε-carbobenzoxy-L-lysine) (1:1 molar ratio), is dissolved in a solvent, such as anhydrous dimethyl formamide (DMF). Two molar equivalents of dicyclohexyl carbodiimide (DCC) and 0.1 molar equivalents of (dimethylamino-pyridine) DMAP may then be added. The reaction is allowed to run. Following conjugation, the polymer solution is diluted, with, for example, chloroform. The copolymer may then be precipitated, with, for example, methanol, and vacuum filtered to remove unconjugated material. The polymer may then be redissolved, in, for example, chloroform, precipitated, in, for example, ether, vacuum filtered and lyophilized.

To expose (deprotect) primary amines, the copolymer is dissolved in hydrogen bromide (HBr), 30 wt % in acetic acid (HBr/HOAc) and stirred. After 1-3 hours, ether may added to the solution and the precipitated polymer is removed, washed, dissolved in chloroform, re-precipitated in ether and lyophilized.

PEG is activated, with, for example, 1,1′-carbonyldiimidazole (CDI). PEG is dissolved, in for example, dioxane. An 8:1 molar excess of CDI is added, and the resulting mixture allowed to stir under argon at 37° C. for 1-3 hours. Unreacted CDI is removed by dialysis. The resulting solution is frozen in liquid nitrogen and freeze-dried for 2-5 days.

A 5:1 molar ratio mixture of excess activated PEG and the copolymer, is dissolved in anhydrous DMF and allowed to stir under argon. An excess of PEG is used to ensure that only one imidazole end group reacted with the pendant amino groups of the polymer, leaving the other imidazole group is available for later conjugation with an RGD moiety. After 1-3 days, the polymer solution is diluted with chloroform and precipitated in methanol. Polymer dissolution and precipitation is repeated two times to ensure the removal of unconjugated PEG. Unconjugated PEG is soluble in methanol and easily removed.

Twenty-five milligrams of a peptide moiety (such as, for example, RGD, RGDS, GRGDS, or GRADSP) may mixed with about 200 mg of the copolymer in about 3 mL of anhydrous DMSO and allowed to stir. After 1-3 days, the polymer solution is diluted with more DMSO, and dialyzed against deionized water for 10-20 hours to remove unconjugated peptide (see, for example, Deng et al., 2007, Polymer 48, 139-149). The RGD conjugated copolymer may then be lyophilized for 2-5 days. After freeze-drying, the polymer is redissolved in DMSO, and dialysis and lyophilization is repeated.

PEG nanoparticles may be fabricated using a solvent evaporation method (see, for example, Hans & Lowman, 2002, Curr. Opin. Solid State Mater. Sci. 6, 319-327). By way of nonlimiting example, PEG conjugated copolymer is dissolved in dichloromethane (DCM). The polymer solution is added dropwise to a vortexing solution of 5% PVA (w/v). The solution may then sonicated, at, for example 38% amplitude for about 30 seconds. After sonication, the emulsion is added to 5% PVA (w/v) and allowed to stir harden for 2-5 hours. Nanoparticles may then collected by centrifugation, washed with deionized water, and freeze-dried for 2-5 days. Then, twenty-five milligrams of an RGD motif containing peptide (such as, for example, RGD, RGDS, GRGDS, or GRADSP) is reconstituted in PBS. This peptide solution may then be added to PEG nanoparticles and allowed to react for 2-5 hours. Following this conjugation of RGD to the PEG imidazole group on the nanoparticle, the nanoparticle/peptide mixture is diluted with deionized water and centrifuged. The supernatant having unconjugated RGD may be discarded. The nanoparticles may then be reconstituted with deionized water and washed two more times by repeating this process. Nanoparticles may the be frozen and freeze-dried for 2-5 days.

### Pharmaceutical Compositions and Therapies

In some embodiments, the compositions comprising a nanoparticle disclosed herein, can be formulated and administered to an animal, preferably a human, in need of reducing or slowing blood loss. In other embodiments, the compositions comprising a nanoparticle disclosed herein, may be formulated and administered to an animal, preferably a human, to facilitate the delivery of a therapeutic agent.

The invention encompasses the preparation and use of pharmaceutical compositions comprising a nanoparticle as described herein. Such a pharmaceutical composition may consist of a nanoparticle alone, in a form suitable for administration to a subject, or the pharmaceutical composition may comprise a nanoparticle and one or more pharmaceutically acceptable carriers, one or more additional ingredients, one or more pharmaceutically acceptable therapeutic agents, or some combination of these. The therapeutic agent may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the therapeutic agent may be combined and which, following the combination, can be used to administer the therapeutic agent to a subject.

As used herein, the term “physiologically acceptable” ester or salt means an ester or salt form of the therapeutic agent which is compatible with any other ingredients of the pharmaceutical composition, which is not deleterious to the subject to which the composition is to be administered.

The methods of treatment of the invention comprise administering a therapeutically effective amount of a nanoparticle of the invention, such as a synthetic platelet or a therapeutic agent delivery vehicle, to a subject in need thereof. It should be understood, that the methods of treatment of the invention by the delivery of a synthetic platelet include the treatment of subjects that are already bleeding, as well as prophylactic treatment uses in subjects not yet bleeding. In a preferred embodiment the subject is an animal. In a more preferred embodiment the subject is a human.

The present invention should in no way be construed to be limited to the synthetic platelets described herein, but rather should be construed to encompass any synthetic platelets, both known and unknown, that diminish or reduce bleeding or blood loss.

The formulations of the pharmaceutical compositions described herein may be prepared by any method known or hereafter developed in the art of pharmacology. In general, such preparatory methods include the step of bringing a nanoparticle comprising an therapeutic agent into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, animals including commercially relevant animals such as cattle, pigs, horses, sheep, cats, and dogs, birds including commercially relevant birds such as chickens, ducks, geese, and turkeys.

Pharmaceutical compositions that are useful in the methods of the invention may be administered, prepared, packaged, and/or sold in formulations suitable for parenteral, oral, rectal, vaginal, topical, transdermal, pulmonary, intranasal, buccal, ophthalmic, or another route of administration. Other contemplated formulations include projected nanoparticles, liposomal preparations, resealed erythrocytes containing the therapeutic agent, and immunologically-based formulations.

The compositions of the invention may be administered via numerous routes, including, but not limited to, parenteral, oral, rectal, vaginal, topical, transdermal, pulmonary, intranasal, buccal, or ophthalmic administration routes. The route(s) of administration will be readily apparent to the skilled artisan and will depend upon any number of factors including the type and severity of the disorder being treated, the type and age of the veterinary or human patient being treated, and the like.

Parenteral administration of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition on or through a surgical incision, by application of the composition on or through a tissue-penetrating non-surgical wound, and the like. In particular, parenteral administration is contemplated to include, but is not limited to, cutaneous, subcutaneous, intraperitoneal, intramuscular, intrasternal injection, intravenous, and intra-arterial.

Formulations of a pharmaceutical composition suitable for parenteral administration comprise the therapeutic agent combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles. Such formulations may further comprise one or more additional ingredients including, but not limited to, suspending, stabilizing, or dispersing agents. In one embodiment of a formulation for parenteral administration, the therapeutic agent is provided in dry (i.e. powder or granular) form for reconstitution with a suitable vehicle (e.g. sterile pyrogen free water) prior to parenteral administration of the reconstituted composition.

Pharmaceutical compositions that are useful in the methods of the invention may be administered systemically in oral solid formulations, ophthalmic, suppository, aerosol, topical or other similar formulations. In addition to the compound such as heparin sulfate, or a biological equivalent thereof, such pharmaceutical compositions may contain pharmaceutically-acceptable carriers and other ingredients known to enhance and facilitate therapeutic agent administration. Other possible formulations, such as nanoparticles, liposomes, resealed erythrocytes, and immunologically based systems may also be used to administer compounds according to the methods of the invention.

The pharmaceutical compositions of the present invention may also be formulated so as to provide slow, prolonged or controlled release of therapeutic agent using, by way of non-limiting examples, polymer matrices, gels, hydrogels, permeable membranes, osmotic systems, multilayer coatings, and/or nanoparticles.

In general, a controlled-release preparation is a pharmaceutical composition capable of releasing the therapeutic agent at a desired or required rate to maintain constant pharmacological activity for a desired or required period of time. Such dosage forms provide a supply of a drug to a body during a particular period of time and thus maintain systemic, regional or local drug levels in the therapeutic range for a more prolonged period of time than conventional non-controlled formulations.

U.S. Pat. No. 5,674,533 discloses controlled-release pharmaceutical compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 describes a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,354,566 discloses a controlled-release powder that contains the therapeutic agent. U.S. Pat. No. 5,733,566, describes the use of polymeric microparticles that release antiparasitic compositions.

The controlled-release of the therapeutic agent may be stimulated by various inducers, for example pH, temperature, enzymes, water, or other physiological conditions or compounds. Various mechanisms of drug release exist. For example, in one embodiment, the controlled-release component may swell and form porous openings large enough to release the therapeutic agent after administration to a patient. The term “controlled-release component” in the context of the present invention is defined herein as a compound or compounds, such as polymers, polymer matrices, gels, hydrogels, permeable membranes, and/or nanoparticles, that facilitate the controlled-release of the therapeutic agent in the pharmaceutical composition. In another embodiment, a component of the controlled-release system is biodegradable, induced by exposure to the aqueous environment, pH, temperature, or enzymes in the body. In another embodiment, sol-gels may be used, wherein the therapeutic agent is incorporated into a sol-gel matrix that is a solid at room temperature. This matrix is implanted into a patient, preferably an animal, having a body temperature high enough to induce gel formation of the sol-gel matrix, thereby releasing the therapeutic agent into the patient.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the activity. The amount of the activity is generally equal to the dosage which would be administered to a subject or a convenient fraction of such a dosage such as, for example, one-half or one-third of such a dosage.

The relative amounts of the therapeutic agent, the pharmaceutically acceptable carrier, and any additional ingredients in a pharmaceutical composition of the invention will vary, depending upon the identity, size, and condition of the subject treated and further depending upon the route by which the composition is to be administered. By way of a non-limiting example, the composition may comprise between 0.1% and 100% (w/w) therapeutic agent.

The synthetic platelet compositions of the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal. While the precise dosage administered will vary depending upon any number of factors, including but not limited to, the type of animal, the amount of bleeding being treated, the type of bleeding being treated, the type of wound being treated, the age of the animal and the route of administration. Preferably, the dosage of the compound will vary from about 1 μg to about 50 mg per kilogram of body weight of the animal. More preferably, the dosage will vary from about 10 μg to about 15 mg per kilogram of body weight of the animal. Even more preferably, the dosage will vary from about 100 μg to about 10 mg per kilogram of weight of the animal.

The therapeutic agent delivery compositions of the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal. While the precise dosage administered will vary depending upon any number of factors, including but not limited to, the type of animal, the therapeutic agent being delivered, the type of disorder being treated, the age of the animal and the route of administration. Preferably, the dosage of the compound will vary from about 1 μg to about 50 mg per kilogram of body weight of the animal. More preferably, the dosage will vary from about 10 μg to about 15 mg per kilogram of body weight of the animal. Even more preferably, the dosage will vary from about 100 μg to about 10 mg per kilogram of weight of the animal.

In addition to the therapeutic agent, a pharmaceutical composition of the invention may further comprise one or more additional pharmaceutically active agents.

As used herein, an “oily” liquid is one which comprises a carbon-containing molecule and which exhibits a less polar character than water.

The pharmaceutical compositions may be prepared, packaged, or sold in the form of a sterile injectable aqueous or oily suspension or solution. This suspension or solution may be formulated according to the known art, and may comprise, in addition to the therapeutic agent, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The compound may be administered to an animal as needed. The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### Therapeutic Agent Delivery Compositions

The invention also includes a nanoparticle therapeutic agent delivery vehicle comprising a copolymer containing PLGA, PLL and PEG, as well as methods of making such a nanoparticle therapeutic agent delivery vehicle, as described elsewhere herein. In some embodiments, the copolymer enables the formation of chemical crosslinks in a hydrogel network, via functional groups that undergo radical chain polymerization reaction upon exposure to UV light in the presence of a photoinitiator. In various embodiments, the nanoparticle therapeutic agent delivery vehicle can facilitate the controlled release of a therapeutic agent.

Hydrogels containing a therapeutic agent, such as, for example, CNTF in a nanoparticle, can be used as a therapeutic agent delivery system. Moreover, because they begin as liquid suspensions, cells can be encapsulated within the hydrogel and distributed throughout the hydrogel network immediately, creating a cell culture scaffold containing a therapeutic agent and cell delivery system within the same construct. Furthermore, it appears that the presence of PLL is useful in these PEG-based hydrogels in order to achieve improved cell behavior, such as, by way of non-limiting examples, cell migration and differentiation.

It is an aspect of the invention that nanoparticles comprising a copolymer of PLGA, PLL, and PEG provide an improved therapeutic agent release profile as compared with PLGA alone. By way of non-limiting examples, nanoparticles comprising a copolymer of PLGA, PLL, and PEG have a smaller initial burst and increased release when the polymer begins to degrade. Although not wishing to be bound by any particular theory, this may indicate that the therapeutic agent, such as, for example, hydrophilic CNTF, associates more strongly with the more hydrophilic copolymer than it does with the PLGA alone. As described herein, the hydrogel itself appeared to have little effect on the release profile, indicating that the hydrogel network is not an strong limiting factor in the therapeutic agent release rate.

The therapeutic agent to be delivered by the compositions and methods of the invention, can encapsulated in, attached to, or dispersed within a nanoparticle therapeutic agent delivery vehicle. Selection of a therapeutic agent to be encapsulated within the nanoparticle therapeutic agent delivery vehicle of the present invention is dependent upon the use of the nanoparticle therapeutic agent delivery vehicle and/or the condition being treated and the site and route of administration.

The nanoparticle therapeutic agent delivery vehicle of the invention may be loaded with a therapeutic agent by encapsulating the therapeutic agent in, attaching the therapeutic agent to, or dispersing the therapeutic agent within a nanoparticle therapeutic agent delivery vehicle. Selection of a therapeutic agent to be encapsulated within the nanoparticle therapeutic agent delivery vehicle of the present invention is dependent upon the use of the nanoparticle therapeutic agent delivery vehicle and/or the condition being treated and the site and route of administration. By way of nonlimiting example, the therapeutic agent may be encapsulated with the therapeutic agent delivery vehicle by dissolving the therapeutic agent in a solution containing at least one polymer material, such as PLGA or PLGA or PLGA-b-PLL-g-PEG, which has been dissolved in a solvent, such as DCM solvent and trifluoroethanol (TFE). Then, the mixture may be added dropwise to a stirring solution, for example, PVA solution, that is stirred while the solvent is allowed to evaporate. Then, the nanoparticles encapsulating the therapeutic agent may be washed with deionized water, frozen in liquid nitrogen and lyophilized to isolate the nanoparticles encapsulating the therapeutic agent.

### Kits

The invention also includes a kit comprising a synthetic platelet of the invention and an instructional material which describes, for instance, administering the synthetic platelet to a subject as a therapeutic treatment or a prophylactic treatment use as described elsewhere herein. In an embodiment, this kit further comprises a (preferably sterile) pharmaceutically acceptable carrier suitable for dissolving or suspending the synthetic platelet of the invention. Optionally, the kit comprises an applicator for administering the synthetic platelet.

The invention further includes a kit comprising a nanoparticle therapeutic agent delivery vehicle as described elsewhere and an instructional material which describes, for instance, administering the nanoparticle therapeutic agent delivery vehicle to a subject as a therapeutic treatment or a prophylactic treatment use described elsewhere herein. In one embodiment, this kit further comprises a (preferably sterile) pharmaceutically acceptable carrier suitable for dissolving or suspending the nanoparticle therapeutic agent delivery vehicle of the invention. Optionally, the kit comprises an applicator for administering the nanoparticle therapeutic agent delivery vehicle.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Synthetic Platelets

The Materials and Methods used in this example are now described.

### Materials

Male Sprague Dawley rats (˜180-200 g), obtained from Charles River Laboratories (Wilmington, Mass., USA), were used Poly(lactic-co-glycolic acid)(PLGA) 503H (Resomer® 503H, 50:50 lactic to glycolic acid ratio and a Mₙ ˜25 kDa) was from Boehringer Ingelheim (Ingelheim, Germany). H signifies PLGA terminated with a carboxylic acid group. Poly(s-carbobenzoxy-L-lysine) (molecular weight ˜1000 Da) (PLL) was from Sigma (St. Louis, Mo., USA). Polyethylene glycol) (PEG)(molecular weight ˜1500 and 4600 Da) was from Acros Organics (Geel, Belgium) and Sigma, respectively. Arg-Gly-Asp (RGD) (SEQ ID NO: 1) peptide sequences were from EMD Biosciences (La Jolla, Calif., USA). Peptide sequences include RGD, Arg-Gly-Asp-Ser (RGDS) (SEQ ID NO: 2), Gly-Arg-Gly-Asp-Ser (GRGDS) (SEQ ID NO: 3), and Gly-Arg-Ala-Asp-Ser-Pro (GRADSP) (SEQ ID NO: 4). Collagen I (rat tail) was from BD Biosciences (San Jose, Calif., USA). Deuterated dimethyl sulfoxide (D₆-DMSO) was from Cambridge Isotope Laboratories, Inc. (Andover, Mass., USA). Poly(vinyl alcohol) (PVA) (88 mol % hydrolyzed) was purchased from Polysciences (Warrington, Pa., USA). CMFDA (5-chloromethylfluorescein diacetate) was from Molecular Probes (Eugene, Oreg., USA). Recombinant human factor VIIa (rFVIIa) was from Innovative Research (Novi, Mich., USA). Vector shield with DAPI was from Vector Laboratories (Burlingame, Calif., USA). All other chemicals were A.C.S. reagent grade and other materials were used as received from Sigma.

### PLGA-PLL Synthesis

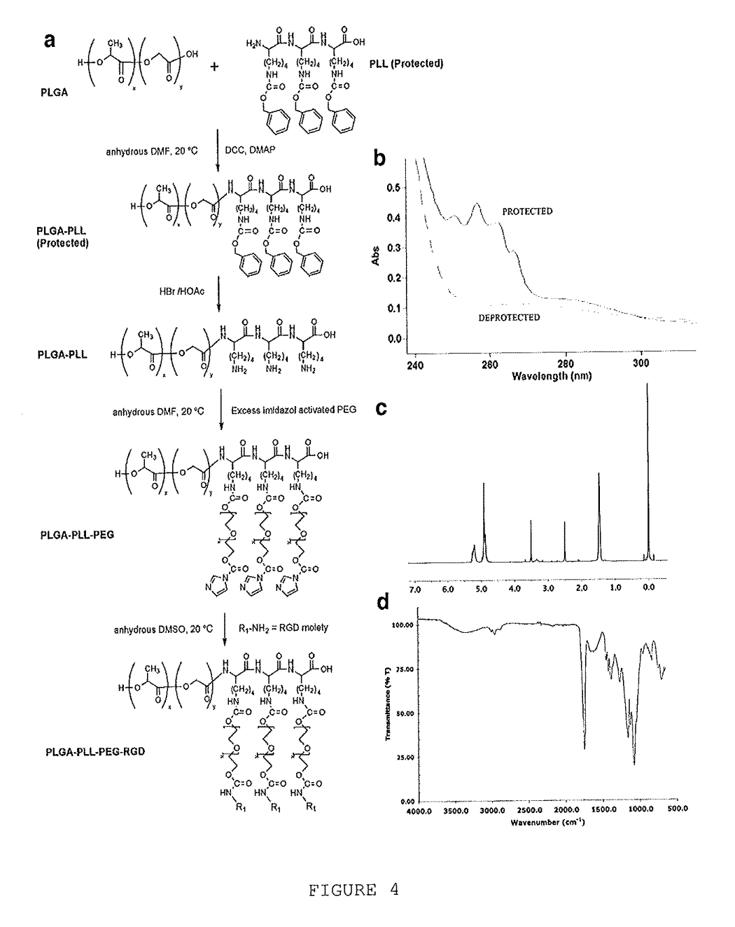

The copolymer PLGA-PLL was synthesized as follows (FIG. 4a). Briefly, PLGA 503H and Poly(ε-carbobenzoxy-L-lysine) (1:1 molar ratio) were dissolved in anhydrous dimethyl formamide (DMF). Two molar equivalents (with respect to PLGA) of dicyclohexyl carbodiimide (DCC) and 0.1 molar equivalents of (dimethylamino-pyridine) DMAP were added. The reaction was allowed to run for 36 hours under argon. Following the conjugation, the polymer solution was diluted with chloroform and filtered to remove N,N′-dicyclohexylurea (DCU), an insoluble by-product of the reaction. The presence of DCU was indicative of successful conjugation. The block copolymer was then precipitated in methanol and vacuum filtered to remove any unconjugated Poly(ε-carbobenzoxy-L-lysine). The polymer was then redissolved in chloroform, precipitated in ether, vacuum filtered and lyophilized for at least 48 hours.

To expose (deprotect) the primary amines of the Poly(ε-carbobenzoxy-L-lysine), ˜1.5 g of the block copolymer was dissolved in hydrogen bromide (HBr), 30 wt % in acetic acid (HBr/HOAc) and allowed to stir. After 1.5 hours, ether was added to the solution and the precipitated polymer was removed. The polymer was washed with ether until an off-white brittle mass was obtained. The mass was then dissolved in chloroform, re-precipitated in ether and lyophilized for 48 hours.

Conjugation/deprotection was verified using ultraviolet-visible spectroscopy (UV-vis) (Cary 50 Bio UV-Vis Spectrophometer, Varian, Palo Alto, Calif., USA). At 257 nM, the protecting carbobenzoxy (CBZ) group can be visualized (FIG. 4b). Presence of this CBZ group prior to deprotection verifies successful conjugation while its later absence is indicative of successful deprotection and thus amine exposure. NMR was also utilized for determining successful conjugation/deprotection of PLGA-PLL (FIG. 4c). NMR spectra were recorded at room temperature in D₆-DMSO on a 400 MHz Bruker (Germany) spectrometer and referenced to tetramethylsilane (TMS) peak (δ=0.0 ppm). The benzene ring peak associated with the CBZ protecting groups (δ=7.3 ppm) is present in the protected PLGA-PLL while successfully removed in the deprotected form (Deng et al., 2007, Polymer 48, 139-149).

### PEG Conjugation to PLGA-PLL

PEG (molecular weight 1500 and 4600 Da) was activated with CDI. Briefly, PEG was dissolved in dioxane at 37° C. An 8:1 (CDI:PEG) molar excess of CDI was added, and the resulting mixture was allowed to stir under argon at 37° C. for 2 hours. Unreacted CDI was removed by dialysis in deionized water for 12 hours. The dialysate was changed every hour. The resulting solution was frozen in liquid nitrogen and freeze-dried for 3 days. The resulting activated PEG was then stored at −20° C.

A mixture of excess activated PEG and PLGA-PLL (5:1 molar ratio PEG:PLGA-PLL) was dissolved in anhydrous DMF and allowed to stir under argon. An excess of PEG was used to ensure that only one imidazole end group reacted with the pendant amino groups of the PLGA-PLL, while the other imidazole group was available for later conjugation to the RGD moiety. After 48 hours, the polymer solution was diluted with chloroform and precipitated in methanol. Unconjugated PEG is soluble in methanol and easily removed. Polymer dissolution and precipitation was repeated two times to ensure the removal of unconjugated PEG.

¹H NMR was also utilized for determining conjugation of PEG to PLGA-PLL. The presence of the ether linkage associated with PEG (δ=3.51 ppm) verified its successful incorporation (FIG. 4c) (Deng et al., 2007, Polymer 48, 139-149).

### RGD Conjugation to PLGA-PLL-PEG

Twenty-five milligrams of the peptide moiety RGD, RGDS, GRGDS, or GRADSP was mixed with 200 mg of PLGA-PLL-PEG in 3 ml of anhydrous DMSO and allowed to stir. After 24 hours, the polymer solution was diluted with more DMSO, and dialyzed against deionized water for 12 hours to remove unconjugated peptide (Deng et al., 2007, Polymer 48, 139-149). The dialysate was changed every hour. The PLGA-PLL-PEG-RGD was then lyophilized for 3 days. Following freeze-drying, the polymer was redissolved in DMSO, and dialysis and lyophilization were repeated.

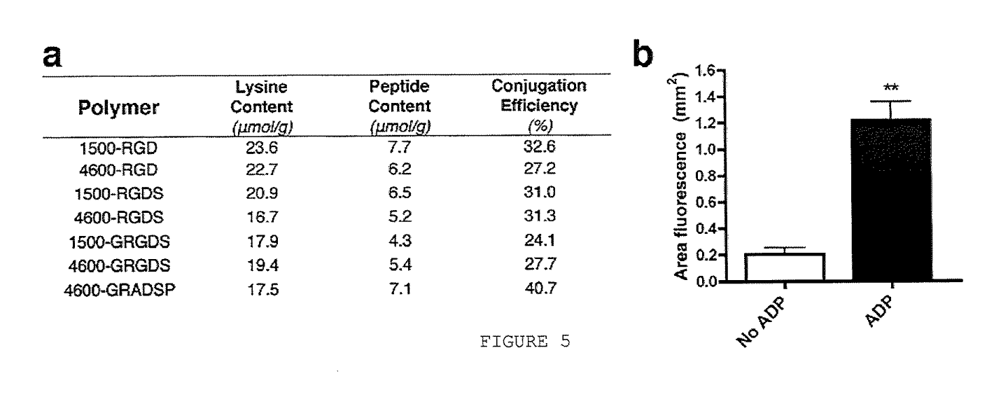

The successful conjugation of RGD was determined using attenuated total reflectance Fourier transform infrared spectroscopy (ATR-FTIR) (Deng et al., 2007, Polymer 48, 139-149; Li et al., 2006, Journal of Biomedical Materials Research Part A 79A, 989-998). Spectra were collected by a Perkin-Elmer Instruments Spectrum One FTIR equipped with a Universal ATR sampling accessory (Waltham, Mass., USA). Each sample was scanned 32 times at a resolution of 1 cm⁻¹. While useful in validating the presence of amide linkages (FIG. 4d), further amino acid (AA) analysis was required to differentiate the PLL and RGD contributions (FIG. 5). For AA analysis, samples were submitted to the W.M. Keck Facility, Yale University. Analyses were carried out on a Beckman Model 7300 ion-exchange instrument. The molar ratio of arginine to lysine was defined as the conjugation efficiency of the RGD moiety to the PLGA-PLL-PEG.

### Nanoparticle Fabrication and Characterization

PLGA-PLL-PEG nanoparticles were fabricated using a solvent evaporation method (Hans & Lowman, 2002, Curr. Opin. Solid State Mater. Sci. 6, 319-327). Two hundred milligrams of polymer (PLGA-PLL-PEG) was dissolved in 2 ml of dichloromethane (DCM). The polymer solution was added dropwise to a 4 ml vortexing solution of 5% PVA (w/v). The solution was then sonicated (Tekmar Sonic Disruptor TM300, Mason, Ohio, USA) for 30 seconds at 38% amplitude. Following sonication, the emulsion was added to 50 ml of 5% PVA (w/v) and allowed to stir harden for 3 hours. Nanoparticles were then collected by centrifugation, washed three times with deionized water, and freeze-dried for 3 days.

Nanoparticle size was determined using dynamic light scattering (DLS) (ZetaPals particle sizing software, Brookhaven Instruments Corp., Holtsville, N.Y., USA) and scanning electron microscopy (SEM) (Phillips XL-30 environmental). Two representative micrographs were taken, and diameters of 40 particles from each image were measured using ImageJ.

### RGD Conjugation to Nanoparticle

Twenty-five milligrams of an RGD motif containing peptide or control peptide (e.g., Arg-Gly-Asp (RGD) (SEQ ID NO: 1); Arg-Gly-Asp-Ser (RGDS) (SEQ ID NO: 2); Gly-Arg-Gly-Asp-Ser (GRGDS) (SEQ ID NO: 3); Gly-Arg-Ala-Asp-Ser-Pro (GRADSP) (SEQ ID NO: 4)) was reconstituted in 3 ml of PBS. This peptide solution was then added to 200 mg of PLGA-PLL-PEG nanoparticles and allowed to react for 3 hours. Following this conjugation of RGD to the PEG imidazole group on the nanoparticle, the nanoparticle/peptide mixture was diluted with deionized water and centrifuged. The supernatant with unconjugated RGD was discarded. Nanoparticles were then reconstituted with deionized water and washed two more times by repeating this process. Nanoparticles were then frozen and freeze-dried for 3 days. Successful conjugation of RGD was determined via AA analysis as previously described. As a control, PLGA-PLL-PEG nanoparticles without imidazole activated PEG were used. These nanoparticles had undetectable levels of RGD present, demonstrating the necessity of the PEG imidazole end groups for peptide incorporation.

### In Vitro Characterization of PLGA-PLL-PEG-RGD Polymer

Ninety-six well plates were coated with PLGA-PLL-PEG-RGD polymer to examine interactions between the polymers and platelets. Briefly, 5 mg of polymer was dissolved in 1.0 ml trifluoroethanol (TFE). One hundred microliters of polymer solution was added to each well of a 96-well plate. By allowing the TFE to evaporate, the wells were effectively coated with the polymer (Deng et al., 2007, Polymer 48, 139-149). Wells were then washed three times with PBS. Following the PBS rinse, 100 μl of PRP with CMFDA fluorescently labeled platelets (5×10⁸ platelets/ml) was added to each well. This was followed by the addition of 10 μl of 100 μM ADP as a proaggregatory stimulus, or PBS as a control. Immediately following ADP/PBS addition, the 96-well plate was agitated for one minute on an orbital shaker (Barnstead International, Dubuque, Iowa, USA) at 180 rpm (Beer et al., 1992, Blood 79, 117-128). Following a 3 minute equilibration, plasma and non-aggregated platelets were gently extracted, and entire wells were imaged from the bottom with a 4× objective at 490 nM/525 nM (excitation/emission) (Olympus IX71 Fluorescent microscope, Center Valley, Pa., USA). Area of fluorescence was then quantified to elucidate the differences in platelet adherence/aggregation (Coller et al. 1992, Journal of Clinical Investigation 89, 546-555). Experiments were performed in triplicate.

### In Vivo Analysis of Hemostasis

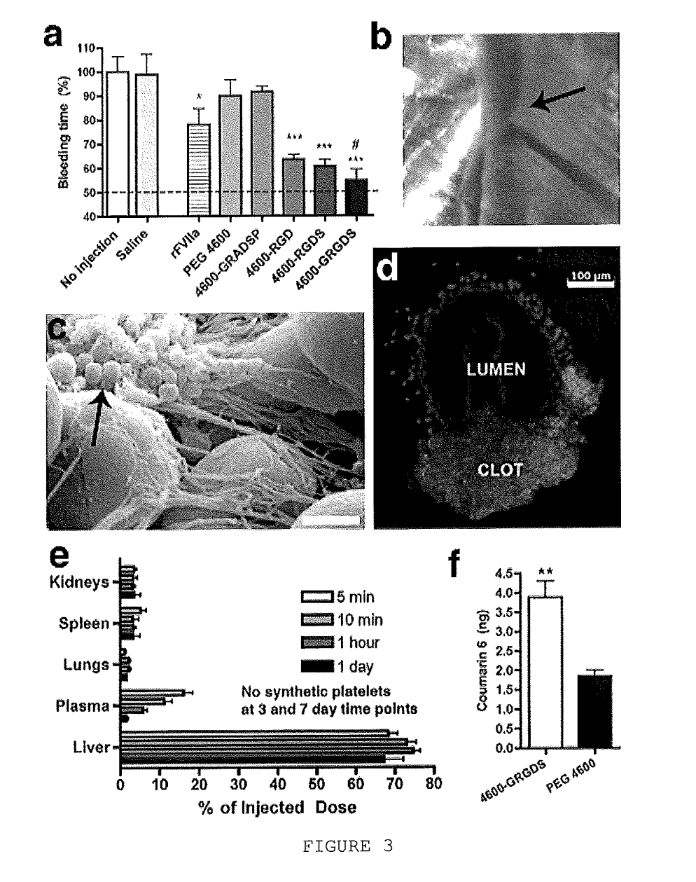

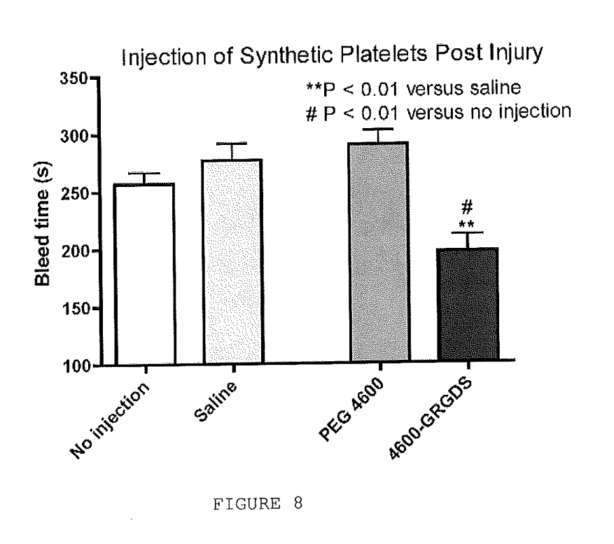

Male Sprague Dawley rats (˜180-200 g), obtained from Charles River Laboratories (Wilmington, Mass., USA), were used. Treatment groups included a sham (injury alone), vehicle (saline) alone, rFVIIa (100 μg/kg), PLGA-PLL-PEG or PLGA-PLL-PEG-RGD nanoparticles at 20 mg/ml. All treatments (excluding sham group) were in 0.5 ml vehicle solution. The surgeon performing the injury was blinded to the treatment groups. Anaesthetized rats were given an intravenous injection via femoral vein cannula, and treatments were allowed to circulate for 5 minutes. In some experiments, a thrombogenic injury was induced in the femoral artery (Fuglsang et al., 2002, Blood Coagulation & Fibrinolysis 13, 683-689) after intravenous administration and circulation (FIG. 3). In other experiments, a thrombogenic injury was induced in the femoral artery before intravenous administration and circulation (FIG. 8). Briefly, a transverse cut made with microscissors encompassing one-third of the vessel circumference resulted in the extravasation of blood. Time required for bleeding to cease for at least 10 seconds was recorded as the bleeding time. Experiments included five rats per group.

### Biodistribution

RGD nanoparticles were fabricated as described herein, with the addition of C6 to the DCM (0.5% w/v). The biodistribution of the RGD nanoparticles was examined following intravenous injection. A 0.5 ml injection (20 mg/ml) of C6 labeled PEG4600-GRGDS nanoparticles was administered via tail vein injection. Biodistribution was examined at 5 minute, 10 minute, 1 hour, 1, 3, and 7 days post injection. At each time point, animals were euthanized and blood, lungs, liver, kidneys and spleen were collected. Blood was centrifuged (180 g for 10 minutes) and 1.0 ml of plasma was extracted. Plasma and organs were then freeze-dried for 3 days and dry organ mass was then determined.

To determine organ C6 content, 50 mg of dry organ was homogenized (Precellys 24 Tissue homogenizer, Bertin Technologies, Montigny-le-Bretonneux, France) in 1.0 ml DMSO. The homogenates were covered and incubated at 37° C. for 6 hours to ensure nanoparticle/C6 dissolution. Homogenates were then centrifuged and 200 μl samples were extracted and analyzed for C6 content. Samples were analyzed at 444/538 nM (excitation/emission) (SpectraMax M5 spectrophotometer, Molecular Devices, Sunnyvale, Calif., USA) for C6 content. A C6 standard curve in DMSO was established with sensitivity to 10 ng/ml. Organs without C6 were analyzed at the same wavelengths to establish background fluorescence. Experiments were performed in triplicate for each time point.

### Biodistribution Following Injury

Biodistribution of C6 labeled, 4600-GRGDS nanoparticles was also examined following the thrombogenic injury to the femoral artery. Nanoparticles were injected intravenous through the femoral vein cannula. Organs were extracted one hour following or immediately after bleeding had stopped. Tissue was processed and C6 was quantified as described. Experiments were performed in triplicate at each time point.

Further analysis included the quantification of C6 associated with the clot following injury. Two groups were analyzed in the femoral artery injury, PEG 4600 and 4600-GRGDS nanoparticles. Five rats were used in each group. Following injury and the cessation of bleeding, the clot was excised and immersed in acetonitrile (ACN) overnight. Samples were centrifuged and C6 content was determined using reverse-phase high performance liquid chromatography (HPLC) (Shimadzu Scientific Instruments, Columbia, Md., USA) with a fluorescence detector and a Nova-Pak® C18, 4 um, 3.9 mm×150 mm column (Waters, Milford, Mass., USA). Mobile phase was prepared as described by Eley et al (Eley et al, 2004, Drug Delivery 11, 255-261), and consisted of ACN:acetic acid (8%) (80:20 v/v) with a flow rate of 1.0 ml/minute. A standard curve for C6 (450/490 nM (excitation/emission) and retention time ˜3.1 minute) was established in ACN with a sensitivity limit of 0.25 ng/ml.

### Clot Visualization

In order to visualize C6 labeled nanoparticles associated within clots following thrombogenic injury, injured vessel segments containing clots were excised and fixed in 10% formalin overnight. Following fixation, clots were either mounted for visualization via SEM, or embedded in OCT. Embedded clots were then cryosectioned to 15 μm cross-sections and mounted with Vector Shield with DAPI. Cross-sections were then visualized with a Zeiss Axiovert 200 microscope (Carl Zeiss Inc., Thornwood, N.Y., USA).

### Fluorescent Labeling of Rat Platelets

Rats were anesthetized with an intraperitoneal (i.p.) injection of ketamine/xylazine (80/10 mg/kg). Following induced anesthesia, blood was obtained via cardiac puncture in a syringe containing 1000 U sodium heparin/ml (in 0.9% saline) solution (anticoagulant solution:blood, 1:9 v/v). To prepare platelet rich plasma (PRP), the collected blood underwent a “soft spin” of 180 g for 10 minutes at 22° C. Platelets were then sedimented by centrifuging the PRP at 1600 g for 5 minutes. PPP was extracted and the remaining platelets were resuspended in Buffer A (140 mM NaCl, 3 mM KCl, 0.5 mM MgCl₂, 5 mM NaHCO₃, 10 mM glucose, and 10 mM Hepes, pH=7.4) (Hoffmeister et al., 2003, Science 301, 1531-1534). Reconstituted platelets were then stained with 10 μM CMFDA (5-chloromethylfluorescein diacetate) (Molecular Probes, Eugene, Oreg.) (2.5 mM stock in DMSO). Platelets were stained for 40 minutes at room temperature, and then centrifuged at 1600 g for 5 minutes. Buffer A was extracted and platelets were reconstituted in platelet poor plasma (PPP) to a final concentration of 5×10⁸ platelets/ml. Platelet concentration was determined using a Beckman Coulter Multisizer 3 (Fullerton, Calif., USA) with a 50 μM diameter aperture based on a sample volume of 100 μl.

### Validation of In Vitro Assay

For the initial validation of the in vitro assay, Collagen I (rat tail) was used. Briefly, 96-well plates were coated by adding 100 μl of 500 μg/ml collagen to each well. Plates were then allowed to sit for 24 hour at 4° C. Wells were then washed three times with PBS to remove inadherent collagen. Following the PBS rinse, 100 μl of PRP with CMFDA fluorescently labeled platelets (5×10⁸ platelets/ml) was added to each well (see Supplementary information CMFDA labeling). This was followed by the addition of 10 μl of 100 μM adenosine diphosphate (ADP) as a proaggregatory stimulus, or PBS as a control. Immediately following ADP/PBS addition, the 96-well plate was agitated for one minute on an orbital shaker (Barnstead International, Dubuque, Iowa, USA) at 180 rpm (Beer et al., 1992, Blood 79, 117-128). Following a 3 minute equilibration, plasma and non-aggregated platelets were gently extracted, and entire wells were imaged from the bottom with a 4× objective at 490 nM/525 nM (excitation/emission) (Olympus IX71 Fluorescent microscope, Center Valley, Pa., USA). Area of fluorescence was then quantified to elucidate the differences in platelet adherance/aggregation (Coller et al. 1992, Journal of Clinical Investigation 89, 546-555).

In Vitro Release of C6 from 4600-GRGDS Nanoparticles

The release of C6 from the 4600-GRGDS nanoparticles was investigated. Briefly, 5 mg of C6 labeled 4600-GRGDS nanoparticles were reconstituted with 1.0 ml of phosphate buffered saline (PBS) in a 1.5 ml eppendorf tubes. Mixtures were then incubated at 37° C. on a rotating shaker. At specific time points (1 hour, 5 hours and 1, 3, and 7 days) the mixture was centrifuged and the supernatant was collected. An equal volume of PBS was then added to replace the withdrawn supernatant and the nanoparticles were resuspended and returned to the shaker. Extracted supernatants were freeze-dried and reconstituted in 1.0 ml DMSO. Samples were then analyzed at 444/538 nM (excitation/emission) (SpectraMax M5 spectrophotometer, Molecular Devices, Sunnyvale, Calif., USA) for C6 content. A C6 standard curve in DMSO was established with sensitivity to 10 ng/ml.

### Surgical Preparation for Femoral Artery Injury

Rats were initially anesthetized with an intraperitoneal injection of ketamine/xylazine and placed in a supine position on a heat pad. Body temperature was maintained at 37° C. An incision was made from the abdomen to the knee on the left hindlimb. Following exposure of the femoral vein, polyethylene tubing (PE 10) was used as a catheter and inserted into the femoral vein. Sutures secured the catheter, the cavity was closed, and the skin was sutured. The canulated vein was later used for the intravenous administration of anesthetics and treatment groups.

Following canulation, a similar incision was made on the right hindlimb, and the femoral vessels were exposed. A portion of the femoral artery was then isolated from the surrounding connective tissue by placing a small piece of aluminum foil between the vessel and the underlying tissue. Once the vessel was isolated, the cavity was irrigated (5 ml/minute) with 0.9% NaCl irrigation fluid (Braun Medical Inc., Irvine, Calif., USA) at 37° C. Following a 10 minute equilibration period, a treatment group was administered intravenous through the cannulated femoral vein over 3 minutes.

The results of this example are now described.

### Synthesis and Characterization of the Synthetic Platelets

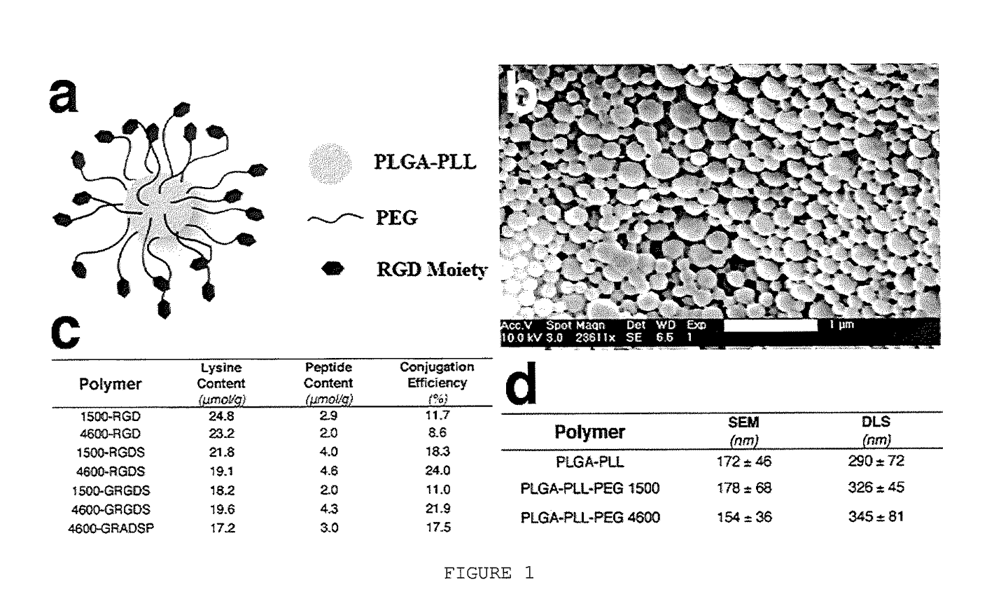

Synthetic platelets were synthesized comprised of poly(lactic-co-glycolic acid)-poly-L-lysine (PLGA-PLL) block copolymer cores with conjugated polyethylene glycol (PEG) arms terminated with RGD functionalities (FIG. 1a). ¹H-NMR demonstrated successful conjugation of PEG to PLGA-PLL (FIG. 4c). Nanoparticles were fabricated using a single emulsion solvent evaporation technique (Hans & Lowman, 2002, Curr. Opin. Solid State Mater. Sci. 6, 319-327), which resulted in core diameters of approximately 170 nM based on scanning electron microscopy (SEM) (FIG. 1b, d). Following fabrication, nanoparticles were analyzed using ¹H-NMR to confirm that the conjugated PEG was present. The successful conjugation of RGD to PLGA-PLL-PEG nanoparticles (also to PLGA-PLL-PEG for in vitro analysis) was determined using amino acid (AA) analysis. RGD conjugation efficiency was independent of both the PEG molecular weight and peptide sequence (i.e. RGD versus GRGDS) (FIG. 1c). Synthetic platelets have an average RGD motif containing peptide content of 3.3±1.1 μmol/g (mean±SD) (FIG. 1c), which corresponds to a conjugation efficiency of 16.2±5.9% (mean±SD) (˜600 RGD moieties/synthetic platelet). While cores are approximately 170 nM in diameter for all of the preparations (FIG. 1d), the hydrodynamic diameter of the spheres, determined by dynamic light scattering (DLS), increased with increasing PEG molecular weight (FIG. 1d). The SEM and DLS results suggest that a surface enrichment of PEG arms exists, and in a hydrated environment PEG arms extend to create a PEG ‘corona’ on the nanoparticle surface (Gref et al., 2000, Colloids and Surfaces B-Biointerfaces 18, 301-313). Based on this, under hydrated conditions, the surface proximity of the conjugated RGD motif containing peptide varies as a function of PEG molecular weight.

### In Vitro Characterization of the Interaction Between Polymers and Activated Platelets

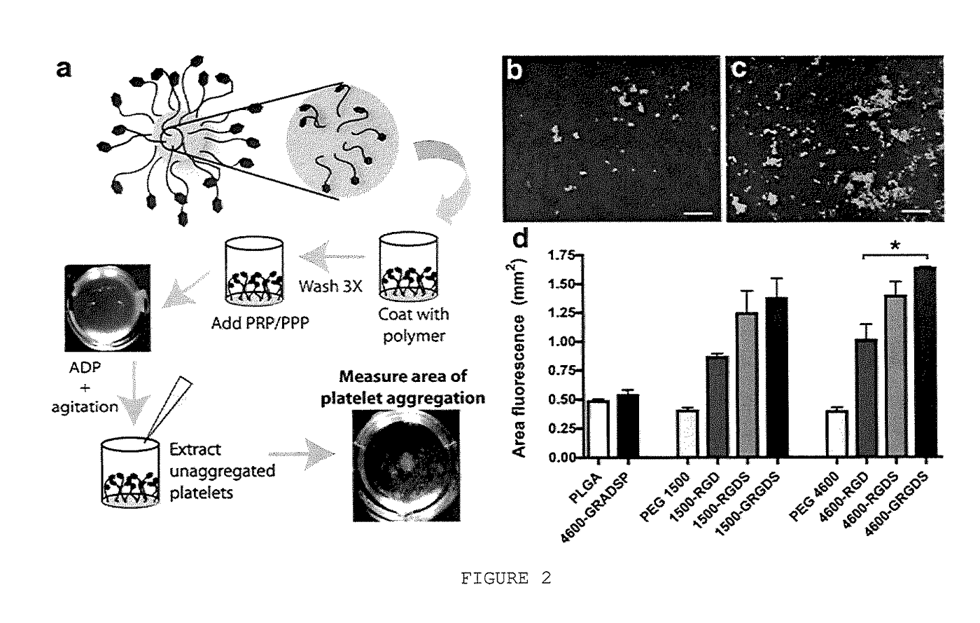

The surface proximity of the RGD functionality has been shown to impact interactions with activated platelets (Beer et al., 1992, Blood 79, 117-128). To investigate the optimal PEG arm length, as well as to identify the most appropriate peptide sequence, an in vitro assay was adapted to elucidate the role of surfaces on platelet aggregation and adhesion (FIG. 2a) (Beer et al., 1992, Blood 79, 117-128). Following validation of the assay with collagen (FIG. 5b), the surface of a 96-well plate was coated with variants of the PLGA-PLL-PEG-RGD polymer (FIG. 4a). Using 5-chloromethylfluorescein diacetate (CMFDA)-labeled platelets and the proaggregatory stimulant adenosine diphosphate (ADP), the effects of the PEG length and RGD functionality on platelet aggregation and adhesion was examined (FIG. 2b, c). Polymer comprised of PEG (molecular weight 4600 Da) and GRGDS (abbreviated as 4600-GRGDS) induced the greatest platelet adhesion and aggregation (FIG. 2d). It has been reported that the incorporation of the RGD moiety can influence cellular interactions with biomaterials (Herselet et al., 2003, Biomaterials 24, 4385-4415), and the proximity of the moiety plays a strong role in these interactions (Beer et al., 1992, Blood 79, 117-128; Ebara et al., 2008, Biomaterials 29, 3650-3655). It was observed that increasing the molecular weight of the PEG led to greater aggregation and adhesion of the activated platelets. Activated platelets bind to RGD through the specific ligand-receptor interactions between RGD and the GP IIb-IIIa receptor expressed on activated platelets (Pytela et al., 1986, Science 231, 1559-1562). The observation that platelet aggregation was greater for PEG 4600 as compared with PEG 1500 supports previous conclusions that RGD proximity influences platelet interactions. Although not wishing to be bound by any particular theory, it is hypothesized that these distances established by PEG molecular weight facilitate RGD/GP IIb-IIIa binding (Beer et al., 1992, Blood 79, 117-128).

The conformation of the RGD motif containing peptide can play a role in the binding of the activated platelets. RGD polymers had the weakest adhesive properties, while GRGDS polymers had the greatest (4600-GRGDS vs. 4600-RGD, FIG. 2d). The data suggest that an increase in the activated platelet's affinity for the GRGDS moiety resulted in an increase in platelet adhesion to the polymer. Similar findings have been reported with cell attachment assays for other cell types (Ebara et al., 2008, Biomaterials 29, 3650-3655). It has been reported that the flanking amino acids influence integrin affinity for the RGD motif (Pierschbacher & Ruoslahti, 1984, Nature 309, 30-33), thereby presenting a more active conformation for binding (Pierschbacher & Ruoslahti, 1987, Journal of Biological Chemistry 262, 17294-17298), and leading to increased cellular attachment (Hirano et al., 1993, Journal of Biomaterials Science-Polymer Edition 4, 235-243). Control experiments verified that the PEG alone, and the scrambled peptide, 4600-GRADSP, were the same as the PLGA-only group, inducing only minimal adhesion and aggregation.

In certain embodiments, activated platelets bind specifically to the synthetic platelets, to avoid nonspecific binding or induced platelet activation which could lead to adverse concomitant thrombotic events, including embolism, and stroke. It was found that non-activated platelets did not bind to any of the PLGA-PLL-PEG-RGD polymers tested. Moreover, platelets did not activate without the addition of ADP. In fact, the polymers did not induce platelet adhesion, even with agitation, except when ADP was added. Furthermore, the materials used to fabricate synthetic platelets do not activate endogenous platelets, and unactivated platelets do not bind, suggesting that the materials are unlikely to induce non-specific platelet binding or activation on their own.

### In Vivo Analysis of Bleed Time and Biodistribution

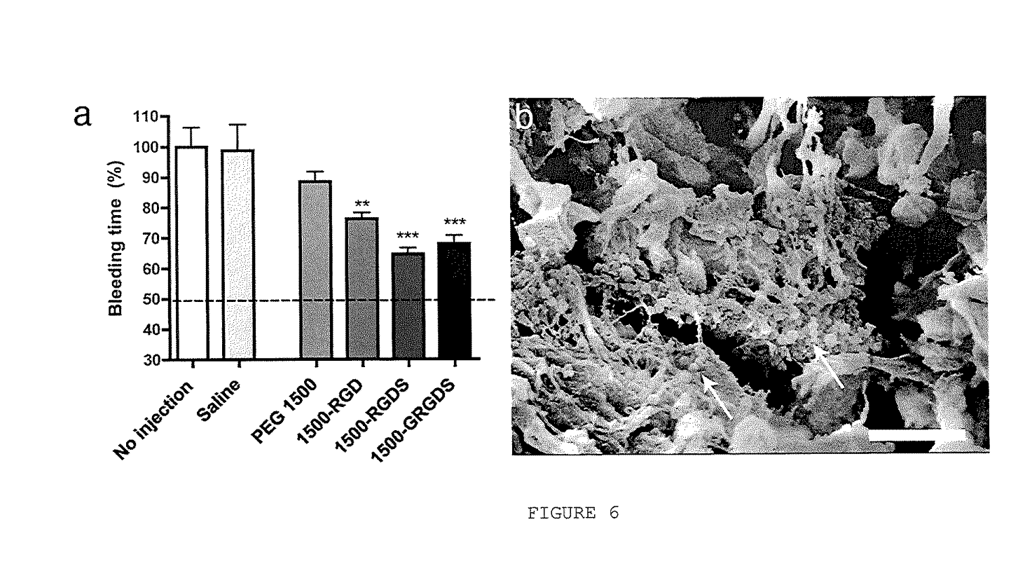

As described herein, the synthetic platelets were tested for safety and hemostatic efficacy in an in vivo major femoral artery injury model (FIG. 3b—injury after administration) (FIG. 8—injury before administration (Fuglsang et al., 2002, Blood Coagulation & Fibrinolysis 13, 683-689). As with the in vitro analysis, the 4600-GRGDS had the greatest hemostatic effect when injected prior to injury (FIG. 3a and FIG. 6a). An injury alone (no injection) resulted in a bleed time of 240±15 seconds (mean±SE). A 20 mg/ml intravenous injection of 4600-GRGDS reduced the bleed time to nearly half (131±11 seconds (mean±SE)). The observed trends in bleed times suggests that the hemostatic attributes of the synthetic platelets are influenced by both the PEG molecular weight as well as the RGD variant (FIG. 3a and FIG. 6a), which are consistent with the in vitro results. Furthermore, 4600-GRGDS synthetic platelets stored in a lyophilized state at room temperature for 2 weeks, maintained their hemostatic properties, suggesting that storage in a clinic, as well as on an ambulance or in a portable medical bag, would be a viable option for applications in the field. To validate the synthetic platelets as a hemostatic agent, bleed times following synthetic platelet administration were compared with bleed times following the injection of recombinant human factor VIIa (rFVIIa). rFVIIa has proven clinically relevant in various instances with surgery and trauma associated bleeding (Martinowitz et al., 2001, Journal of Trauma-Injury Infection and Critical Care 51, 431-438), and is the current standard of care for uncontrolled bleeding (Benharash & Putnam, 2005, (Southeastern Surgical Congress, Santa Barbara, Calif.) 776-780). While a bolus injection of rFVIIa (100 μg/kg) significantly reduced bleed times (187±16 seconds (mean±SE)), 4600-GRGDS synthetic platelets had a significantly greater effect, leading to an approximately 25% further reduction in bleed time as compared to rFVIIa (FIG. 3a).

To determine how the synthetic platelets were physically associated with the clots, injured vessel segments were excised, and SEM micrographs were taken of the clots (FIG. 3c and FIG. 6b). The synthetic platelets are not only intimately associated with the fibrin mesh (FIG. 3c) but are also distributed throughout the clot (FIG. 6b). Cross sections of clots corroborate this observation. Synthetic platelets (4600-GRGDS) were labeled by encapsulating coumarin 6 (C6), a fluorochrome commonly used for biodistribution studies (Eley et al, 2004, Drug Delivery 11, 255-261). Following intravenous administration, C6-labelled synthetic platelets were observed throughout the clot (FIG. 3d). The amount of synthetic platelets within the clots was quantified using HPLC and compared with the intravenous administration of C6 labeled PEG 4600 nanoparticles that did not contain the RGD functionality. While PEG 4600 nanoparticles were entrapped in the clots following injury, clots from animals that received synthetic platelets had more than double the number of particles compared to PEG 4600 nanoparticles (3.9±0.4 and 1.8±0.2 ng of C6, respectively (mean±SE)) (FIG. 3f). However, PEG 4600 nanoparticles had no significant effect on bleeding time (FIG. 3a). Although not wishing to be bound by any particular theory, this data suggests that the synthetic platelets may interact preferentially with activated platelets due to RGD functionalization, and thereby actively halt bleeding, instead of indirectly inducing platelet flocculation).

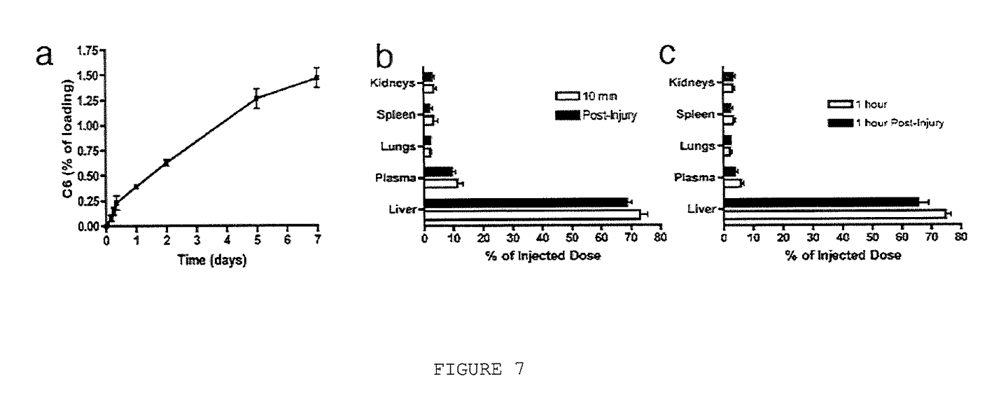

The biodistribution and clearance of C6 labeled synthetic platelets was evaluated for up to 7 days post intravenous injection. Less than 0.5% of the loaded C6 fluorochrome label was released from nanoparticles after 24 hours, and only approximately 1.5% was released after 7 days (FIG. 7a), indicating that all of the C6 fluorescence was associated with synthetic platelets. A characteristic distribution of synthetic platelets was observed that is consistent with intravenous nanoparticle administration (FIG. 3e) (Chambers & Mitragotri, 2007, Experimental Biology and Medicine 232, 958-966). Within 5 minutes of injection, 68.3% of the injected particles were found in the liver, 16.1% were in the blood, and minimal accumulations were seen in the kidneys, lungs and spleen (<3.6%, 2.2%, and 5.2%, respectively). At 3 and 7 days post injection, no C6 was detected, suggesting that all synthetic platelets had been cleared from circulation. Furthermore, no adverse effects were seen in any of the animals, at all time points. The biodistribution of synthetic platelets immediately following a femoral artery injury, and 1 hour post injury, was also examined (FIG. 7b,c). For both time points, tissue distribution was similar for the injured and uninjured animals (equivalent to 10 minutes and 1 hour time points in uninjured animals). This suggests that even following a severe injury and with circulating activated platelets, the unbound synthetic platelets will be effectively cleared within 24 hours.

Ease of administration, stability, non-immunogenicity, and hemostatic efficacy without pathological thrombogenicity, are preferred properties of the synthetic platelets of the invention. Each of the materials used in the synthesis, PLGA, PEG, and the RGD moiety, have been approved in other devices by the FDA (Jain, 2000, Biomaterials 21, 2475-2490; Harris, 1985, Journal of Macromolecular Science-Reviews in Macromolecular Chemistry and Physics C25, 325-373; Kleiman et al., 2000, Circulation 101, 751-757). The choice of nanoparticles, with a PEG enrichment on the surfaces, facilitated both its administration and residence in the circulation (Klibanov et al., 1990, Febs Letters 268, 235-237). By using a small, synthetic peptide sequence (RGD as compared to a protein), problems with immunogenicity and stability are reduced or eliminated. Furthermore, the cost of smaller active peptide sequences is more amenable to translation. The track record of the materials and design for safety, coupled with lack of platelet activation and rapid clearance exhibited here, suggests that the synthetic platelets will be safe. The significant improvement in efficacy in halting bleeding as compared to established treatments (i.e., rFVIIa), demonstrates that these novel synthetic platelets are an ideal candidate for translation into not only the clinic, but also for applications in the field, for example, on an ambulance or at the site where the trauma occurred.

### Statistical Analysis

Data were analyzed using a one-way analysis of variance (ANOVA) followed by the Student-Newman-Keuls test for determining differences between groups. Differences were accepted as statistically significant with P<0.05. Student's t test was used for clot associated C6 comparison and in vitro assay validation with collagen.

### Example 2

### Therapeutic Agent Delivery

The Materials and Methods used in this example are now described.

### Materials

PLGA Resomer 502H (Mn ˜10 k Da, 50:50 lactide:glycolide) was obtained from Boehringer Ingelheim GmbH (Germany). Bovine serum albumin (BSA) and the surfactant sodium bis (2-ethyl-1-hexyl)sulfosuccinate or Aerosol OT (AOT) were purchased from Fisher Chemicals (Fair Lawn, N.J.). Poly(vinyl alcohol) (PVA) with Mw ˜25 k Da was obtained from PolySciences (Warrington, Pa.). Recombinant human ciliary neurotrophic factor (CNTF) with BSA carrier and the Enzyme-Linked Immunosorbent Assay (ELISA) kit were purchased from R&D Systems (Minneapolis, Minn.). Poly(ε-carbobenzoxy-L-lysine) (CBZ-PLL; MW 1000 Da by LALLS) was obtained from Sigma. Poly(ethylene glycol) (PEG, linear, Mn ˜4 kDa) and PEG monomethyl ether (linear, Mn ˜5 kDa) were obtained from Polysciences, Inc. (Warrington, Pa.). Poly(L-lysine) (PLL, MW 1250 Da) was from Sigma. The photoinitiator 2-Hydroxy-1-[4-(2=hydroxyethoxy)phenyl]-2-methyl-1-propanone (Irgacure 2959) was obtained from Ciba Specialty Chemicals (Tarrytown, N.Y., USA) and SpectraPor dialysis membranes (MWCO 1 kDa, 8 kDa) were purchased from Spectrum Laboratories (Rancho Dominguez, Calif., USA). Dimethylaminopyridine (DMAP), dicyclohexyl carbodiimide (DCC), anhydrous dimethylformamide (DMF), hydrogen bromide, 30 wt % in acetic acid (HBr/HOAc), and N,N′-carbonyldiimidazole (CDI) were obtained from Aldrich. All other chemicals were reagent grade. Alexa Fluor 647 secondary antibodies were purchased from Molecular Probes (Eugene, Oreg.). VECTASHIELD mounting medium with 4′-6-diamidino-2-phenylindole (DAPI) was purchased from Vector (Burlingame, Calif.).

### Synthesis of Novel PLGA-b-PLL-g-(PEG Acrylate) Copolymer: Coupling of PLGA-b-PLL Diblock Copolymer

As previously described (Hynes et al., 2007, J Biomater Sci Polym Ed 18:1017-30), PLGA and CBZ-PLL were dissolved in DMF under argon. Briefly, a solution of two molar equivalents (with respect to the number of carboxylic acid groups in the PLGA) of DCC and 0.1 molar equivalent of DMAP in DMF was added to the polymer solution with constant stirring for 48 hours under argon. The solution was diluted by the addition of chloroform, and the polymer product was precipitated in methanol, isolated by vacuum filtration, redissolved in chloroform, reprecipitated in diethyl ether, and lyophilized for at least 24 hours, yielding the block copolymer with all ε-amines of the PLL still protected by the carbobenzoxy (CBZ) protecting group. The reaction efficiency was determined by the concentration of the CBZ ring as measured by UV-visible spectroscopy.

For deprotection, the protected copolymer was dissolved in HBr in acetic acid under argon and stirred for 90 minutes. The polymer was then precipitated with diethyl ether and washed several times with ether. The final, deprotected product with HBr washed away was then redissolved in chloroform, precipitated in ether, vacuum-filtered, and lyophilized for at least 24 hours. The deprotection was verified by the lack of CBZ rings seen by UV-visible spectroscopy.

### Formation of PEG Monoacrylate

PEG functionalized with acrylate as described previously (Lavik et al., 2001, J Biomed Mater Res 58:291-294). Briefly, PEG was dissolved in anhydrous dichloromethane (DCM) under argon. Nine molar equivalents of triethylamine (TEA) with respect to the number of PEG hydroxyls participating in the reaction (half of all PEG hydroxyls) were added to the reaction flask. Acryloyl chloride was added dropwise at a ratio of 2.3:1 acryloyl chloride-to-hydroxyls. This was stirred under argon for 24 hours. The product was precipitated into cold diethyl ether, vacuum filtered, redissolved in filtered deionized water, and dialyzed against water for 48 hours. The solution was frozen in liquid nitrogen and lyophilized until dry to yield PEG monoacrylate. The presence of acrylate groups was verified by ¹H-NMR on a Bruker 500 MHz NMR spectrometer (Worcester, Mass., USA) in deuterated chloroform solvent.

### Grafting of Activated PEG Monoacrylate to PLGA-b-PLL

PEG monoacrylate was activated by dissolving the polymer in 1,4-dioxane with 8 molar equivalents of CDI. The reaction mixture was stirred under argon at 37° C. for 2 hours and then dialyzed against water for 8 hours to remove excess CDI. This was then frozen in liquid nitrogen and lyophilized. The activation was verified by ¹H-NMR in deuterated chloroform.

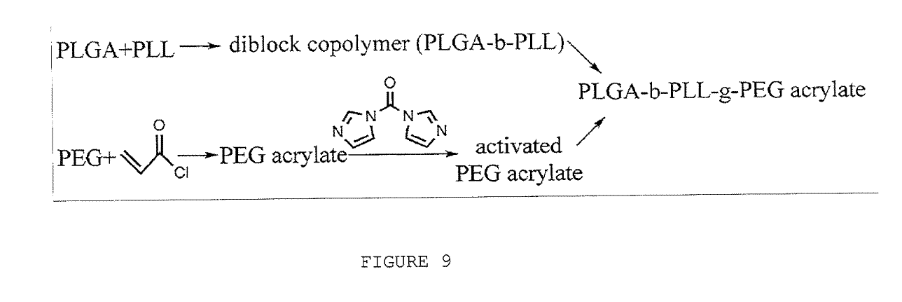

Deprotected PLGA-b-PLL and activated PEG monoacrylate were dissolved together in anhydrous DMF under argon and stirred continuously for 24 hours. The resulting solution was precipitated into diethyl ether, isolated by vacuum filtration, and lyophilized for 24 hours to yield PLGA-b-PLL-g-PEG acrylate (FIG. 9). The reaction was verified by the presence of acrylate protons and absence of imidazole protons as seen by ¹H-NMR.

For controls that do not contain acrylate groups, PEG monomethyl ether was also activated separately with CDI and grafted to PLGA-b-PLL as described above to form PLGA-b-PLL-g-PEG monomethyl ether.

### Preparation of CNTF Nanoparticles

As described previously (Nkansah et al., 2008, Biotech Bioeng 100:1010-9) 300-μL mixture of protein and surfactant (1:20 CNTF:BSA mol/mol, 1:10 protein:AOT mol/mol) were added to a solution consisting of 200 mg polymer (PLGA or PLGA-b-PLL-g-PEG) dissolved in a 6.3-mL mixture of DCM solvent and trifluoroethanol (TFE) cosolvent [1:8, DCM:TFE (v/v)]. The resulting mixture was added drop-wise to 40 mL of stirring 5% PVA solution for cosolvent diffusion and solvent evaporation. This was stirred for 3 hours, then centrifuged and washed three times with deionized water, then frozen in liquid nitrogen and lyophilized to isolate the particles. Blank nanoparticles encapsulating BSA but no CNTF were prepared as controls. All nanoparticles were stored at −20° C.

Protein Release from Nanoparticles

The amount of CNTF released over time was studied as described previously (Sawhney et al., 1993, Macromolecues 26:581-7) by suspending 10 mg of particles in 1 mL of 1× PBS. Tubes were incubated with agitation at 37° C. on a Labquake shaker/rotator. At each time point, tubes were centrifuged and the supernatant removed and stored at −20° C. The particles were resuspended in PBS and replaced in the incubator with agitation. Protein concentrations were determined using standard Enzyme-Linked Immunosorbent Assay (ELISA) protocols. Experiments were done in triplicate.

For nanoparticles encapsulated within hydrogels, hydrogels were incubated at 37° C. in 1× PBS, and surrounding liquid was removed from around the hydrogel at each time point. Samples were stored and protein concentrations determined as above.

### Preparation of Hydrogels: Macromer Synthesis

The hydrogel macromer was either PEG acrylate or PLL-g-PEG acrylate. In both cases, PEG acrylate was prepared as described above; for gels made of PEG acrylate only, twice the amount of acryloyl chloride was used in the acrylation reaction; for gels made of PLL-g-PEG acrylate, monoacrylated PEG was activated with CDI as described above and dissolved in 50 mM sodium bicarbonate buffer (pH 8.2) with PLL, then stirred for 2 hours at room temperature to form the copolymer. This was dialyzed against water for 48 hours with the membrane pore size chosen so that all retained product must contain PLL bound to at least two PEG molecules. This was then frozen in liquid nitrogen and lyophilized (Hynes et al., 2007, J Biomater Sci Polym Ed 18:1017-30).

### Hydrogel Preparation

The photoinitiator Irgacure 2959 was dissolved in MilliQ water at a concentration of 5 mg/mL, keeping the solution from light at all times. The hydrogel macromer (PEG acrylate or PLL-g-PEG acrylate) was dissolved in the photoinitiator solution at 10% (w/v) and placed directly under a UV lamp (365 nM) for 5 minutes. For hydrogels that included nanoparticles, 1% (w/v) nanoparticles were added to the macromer solution and mixed by vortex until suspension appeared homogeneous, and then placed under UV light.

### Rheology

Photopolymerized gels were cut to 20-mm diameter discs. Elastic and viscoelastic moduli were measured using a Rotational Shear Rheometer (AR 1000, TA Instruments, New Castle, Del., USA). Moduli were calculated at constant 10 Pa stress from 0.01 to 100 Hz.

### Culture of NSCs

NSCs were positive for green fluorescent protein (GFP) and were maintained in high glucose DMEM/F12 (1:1) supplemented with 20 ng/mL mouse epidermal growth factor (EGF), N-2, B-27, penicillin/streptomycin, and L-glutamine.

### Characterization of NSC Behavior

To test the response of NSCs cultured near but not directly on or within hydrogels with a CNTF delivery component, NSCs were seeded as neurospheres in chamber slides at a concentration of 5×10⁵ cells/mL. A hydrogel containing acrylated nanoparticles encapsulating an estimated 10 ng of CNTF was added to the culture medium. In other experiments a hydrogel containing either blank (BSA-only) nanoparticles, no nanoparticles, or unencapsulated CNTF at 10 ng/mL was added, and no hydrogel was added in the negative control. Throughout the seven days of the experiment, the migration of NSCs out of the neurospheres was measured using the fluorescence of the GFP NSCs. At the end of seven days, all hydrogels were removed. Cells were fixed for analysis in 10% formalin for 1 hour and rinsed in 1× PBS. Differentiation was quantified by immunocytochemistry as described below.

To test the behavior of NSCs encapsulated within the photopolymerized hydrogels, NSCs were added to the macromer solution as neurospheres and triturated gently to distribute throughout the solution, then placed under the UV light and cured. The gel was then removed and placed in cell culture medium. NSCs were seeded at a concentration of 5×10⁵ cells/mL. Again, migration was monitored throughout the experiment, and cells were fixed at the end of seven days. The hydrogel was then removed, cryosectioned (40-μm sections), and stained by immunocytochemistry.

### Characterization of NSC Differentiation and Survival by Immunocytochemistry

Samples were blocked (3% normal goat serum, 5% BSA, 0.3% TritonX-100) for 1 hour, then rinsed and incubated with primary antibodies against mouse β-III-tubulin (B3T; Promega; 1:1000), mouse nestin (BD Biosciences; 1:200), rabbit glial fibrillary acidic protein (GFAP; Sigma; 1:160), rabbit oligodendrocyte transcription factor 1 (Olig1; Chemicon; 1:200), or caspase-3 (Sigma, 1:10) for 2 hours. They were then rinsed and incubated with species-specific Alexa Fluor 647 secondary antibodies (Molecular Probes; 1:200) for 1 hour, and rinsed again. Cells in chamber slides were also stained with DAPI (1:35000) for 5 minutes, rinsed again, and stored in 1× PBS at 4° C. Cryosections on slides were covered with coverslips using VECTASHIELD mounting medium with DAPI.

The slides were viewed using a Zeiss Axiovert 200 microscope with a Zeiss Mrc camera, and images were captured through Axiovert 4.0 software. Expression of each marker was quantified by the ratio of the area of fluorescence for each marker to the area of fluorescence for the GFP labeling. Results are expressed as mean±coefficient of variation.

### Results

The results of this example are now described.

### Polymer Synthesis and Characterization: UV-Visible Analysis: PLGA-b-PLL Block Copolymer

UV-visible analysis of the protected copolymer showed as expected (Lavik et al., 2001, J Biomed Mater Res 58:291-294) that there are three distinctive peaks around 257 nM, indicative of the protecting group on CBZ-PLL. When compared with CBZ-PLL alone at the same concentration as would be expected from a 1:1 ratio of PLGA to CBZ-PLL (i.e. 100% efficiency), the coupling efficiency was found to be 42.1±5.1%. After deprotection, no CBZ can be found.

¹H-NMR Analysis: Acrylate Group on PEG Acrylate and PLGA-b-PLL-g-(PEG Acrylate)

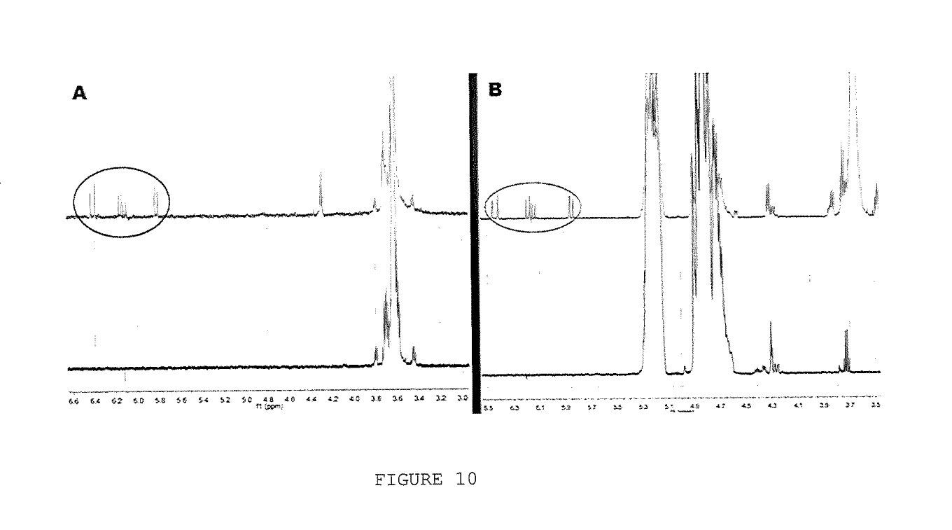

The acrylate group is clearly visible by NMR spectroscopy. The protons in the PEG subunits are ether protons, with a peak at 3.65 ppm. The acrylate protons are visible at 5.87, 6.17, and 6.42 ppm (FIG. 10a). By integrating over the area of each peak, the average number of acrylate peaks on each PEG molecule could be approximated. For hydrogels made of PEG acrylate alone, PEG with 85% of the hydroxyls replaced with acrylate groups was used. For hydrogels made of PLL-g-PEG acrylate, monoacrylated (50% acrylate) PEG was used for the reaction with PLL.

For PLGA, the most prominent peaks are the methyne proton on the lactide subunits (5.21 ppm), the methylene protons on the glycolide subunits (4.80 ppm) and the protons on the methyl group of the lactide subunits (1.58 ppm). The acrylate groups are still detectable after grafting activated PEG acrylate onto PLGA-b-PLL chains (FIG. 10b), and ratios can also be used to determine the efficiency of this grafting reaction, which was calculated to be 72.3±11.7%.

CNTF Release from Nanoparticles, Hydrogels, and Nanoparticle/Hydrogel Composites

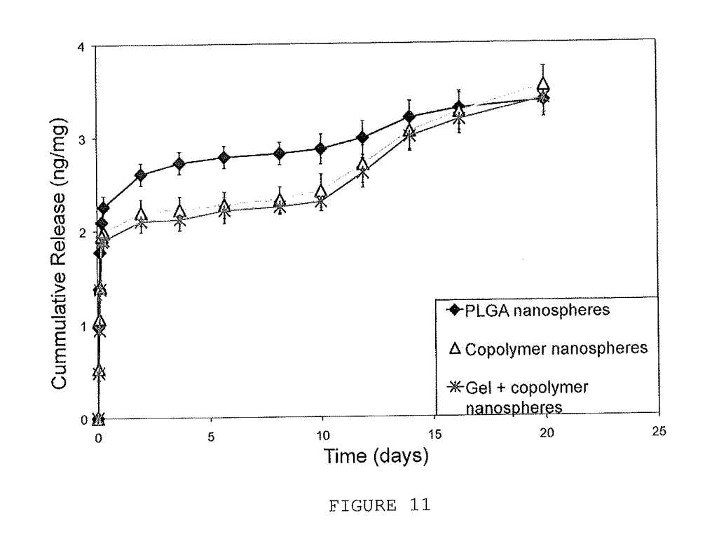

There was an initial burst of CNTF release from all formulations within the first 24 hours from the CNTF adsorbed to the surface or near the surface of the particles. There was also a second phase of release starting after the tenth day. In nanoparticles made with the PLGA-b-PLL-g-PEG copolymer, the burst was reduced slightly and the second phase of release was more robust (FIG. 11). Nanoparticles made of the copolymer that were encapsulated in the hydrogel had an almost identical release profile with nanoparticles separate from the hydrogel.

### Rheology: Mechanical Testing of Hydrogels

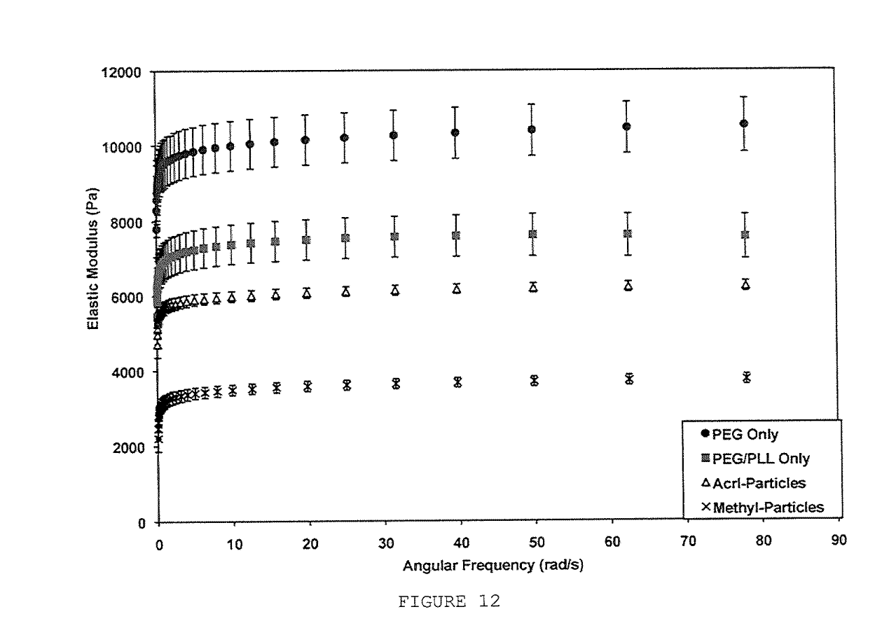

The hydrogels made with PEG acrylate alone had relatively high moduli of up to approximately 10 kPa, while those made with PLL-g-(PEG acrylate) had moduli of approximately 7.5 kPa. The addition of unacrylated nanoparticles made of PLGA-b-PLL-g-(PEG monomethyl ether) tended to cause a decrease in elastic modulus (less than 4 kPa). When the acrylated copolymer is used to make the nanoparticles, the modulus is only reduced to approximately 6 kPa (FIG. 12).

### NSC Behavior: Use of CNTF Nanoparticle/Hydrogel Composites as Therapeutic Agent Delivery Systems

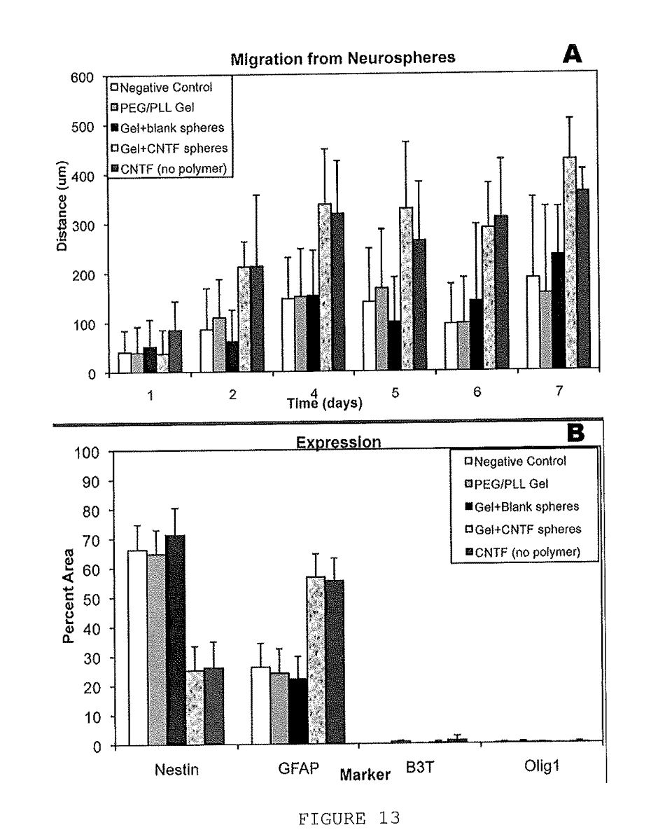

NSCs were seeded in a chamber slide and hydrogel added to the culture medium as a localized therapeutic agent delivery system rather than as a cell culture scaffold. The migration of NSCs out of neurospheres did not show a linear trend, but there was a general trend of increased migration with time, as well as increased migration in the presence of CNTF. PEG, PLGA, and PLL on their own or in combination did not seem to affect migration in the absence of CNTF (FIG. 13a).

These NSCs also showed differentiation toward astrocytes, as evidenced by downregulation of nestin, a neural progenitor marker, and upregulation of GFAP, an astrocytic marker (FIG. 13b).

In one embodiment, the hydrogel/nanoparticle composite described herein can act as a therapeutic agent delivery system. By way of non-limiting example, has described herein, NSCs respond to CNTF delivered from the hydrogel/nanoparticle composite in the same way that would be expected if the cells had been cultured in the presence of CNTF alone without other polymers, indicating that the fabrication of the nanoparticles and hydrogel did not significantly affect the bioactivity of CNTF. Furthermore, the effect of released CNTF on NSC differentiation is consistent with previous studies (Nkansah et al., 2008, Biotech Bioeng 100:1010-9).

Although not wishing to be bound by any particular theory, the data described herein suggest that for the NSCs encapsulated within PEG, or PLL-g-PEG hydro gels, the interaction between the scaffold and the cells affects the cells' behavior and their response to external factors. For example, there is greater migration and differentiation seen when gels are made of the PLL-containing copolymer. Moreover, PEG is known to have the tendency to resist adsorbing proteins (Fu et al., 2003, J Pharm Sci 92:1582-91), which can be a hindrance to cell attachment and movement throughout the environment. This may suggest that the presence of PLL in the hydrogel makes the environment more permissive than if the hydrogel were made of PEG alone. Because PLL has sidechains with free amines that can be protonated in aqueous solution, this may increase the protein adsorption to the material and/or improve the interaction of the cells with the scaffold.

### Encapsulation of NSCs in Photopolymerized Hydrogels

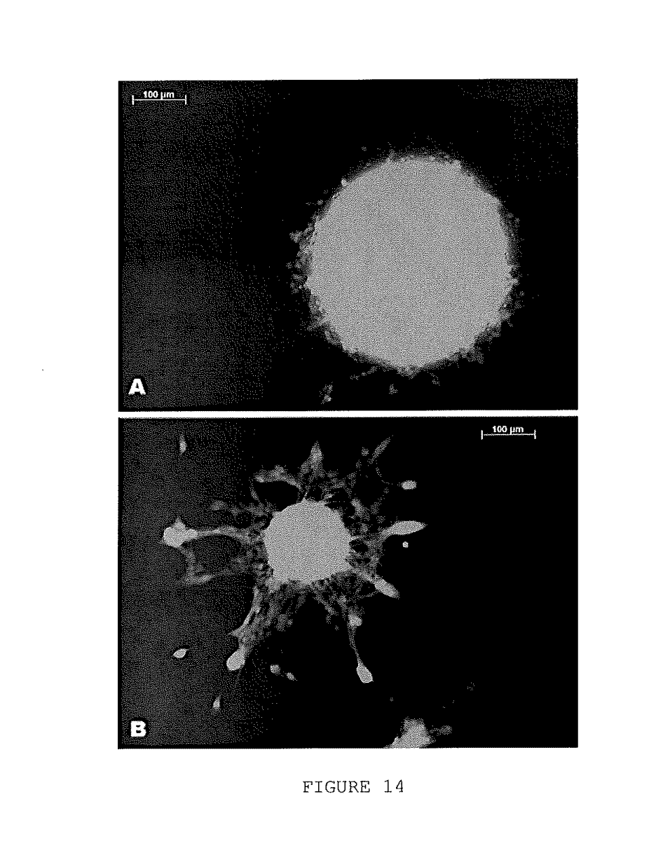

When hydrogels are made of PEG acrylate alone, NSCs show little to no migration out of the neurosphere throughout the experiment (FIG. 14a). When PLL-g-PEG acrylate is used to make the hydrogel, increased migration can be seen from NSCs encapsulated within the gel (FIG. 14b).

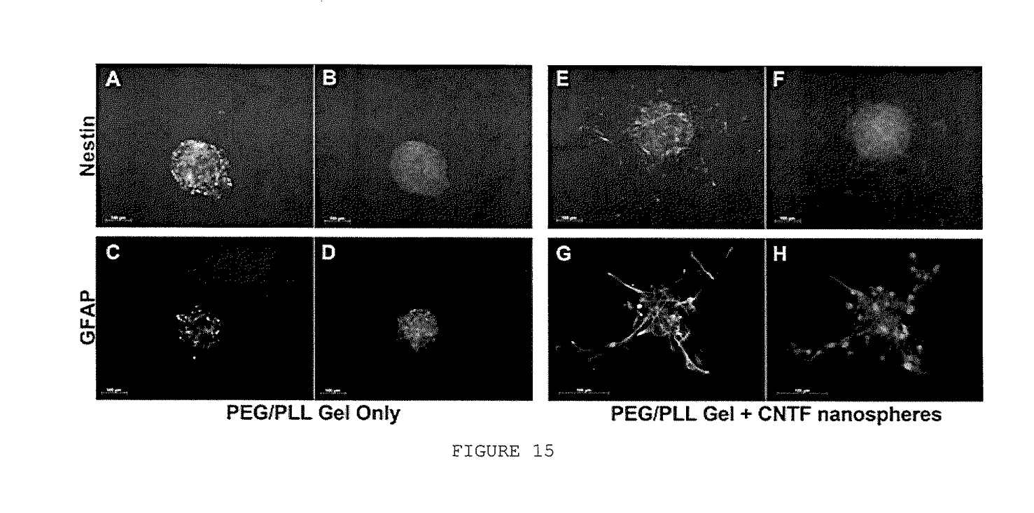

Study of marker expression shows a significant decrease in nestin expression and increase in GFAP (FIG. 15), both of which were found to be statistically insignificant (p>0.05) when using gels made entirely of PEG. In each pair of images, the left shows nestin (A-B, E-F) or GFAP (C-D, G-H) expression and the right shows expression of the protein, cell bodies (GFP), and nuclei (DAPI). NSCs encapsulated within hydrogels without CNTF (A-D) show high nestin and low GFAP expression. NSCs encapsulated with CNTF nanoparticles (E-H) show some downregulation of nestin and marked increase in GFAP expression.

### Other Embodiments

While the invention has been disclosed with reference to specific embodiments, it is apparent that other embodiments and variations of the invention may be devised by others skilled in the art without departing from the true spirit and scope of the invention. The appended claims are intended to be construed to include all such embodiments and equivalent variations.

The disclosures of each and every patent, patent application, and publication cited herein are hereby incorporated herein by reference in their entirety.

What is claimed is:

1. A synthetic platelet composition comprising a polyhydroxy acid polymer, wherein the polyhydroxy acid polymer is conjugated with at least one polyethylene glycol molecule, and wherein the polyethylene glycol molecule is conjugated with at least one RGD motif containing peptide.

2. The composition of claim 1, wherein the polyhydroxy acid polymer comprises at least one of the group selected from poly-lactic-co-glycolic acid and poly-L-lactic acid.

3. The composition of claim 1, wherein the polyhydroxy acid polymer comprises a poly(lactic-co-glycolic acid)-poly-L-lysine (PLGA-PLL) copolymer.

4. The composition of claim 1, wherein the polyethylene glycol molecule is at least one selected from the group consisting of PEG 200, PEG 1000, PEG 1500, PEG 4600 and PEG 10,000.

5. The composition of claim 1, wherein the RGD motif containing peptide is at least one selected from the group consisting of Arg-Gly-Asp (RGD) (SEQ ID NO: 1), Arg-Gly-Asp-Ser (RGDS) (SEQ ID NO: 2), and Gly-Arg-Gly-Asp-Ser (GRGDS) (SEQ ID NO: 3).

6. The composition of claim 1, wherein the synthetic platelet composition further comprises a pharmaceutically acceptable carrier.

7. A synthetic platelet composition comprising a poly(lactic-co-glycolic acid)-poly-L-lysine (PLGA-PLL) copolymer, wherein the a poly(lactic-co-glycolic acid)-poly-L-lysine (PLGA-PLL) copolymer is conjugated with at least one PEG 4600 molecule, and wherein the PEG 4600 molecule is conjugated with at least one Gly-Arg-Gly-Asp-Ser (GRGDS) (SEQ ID NO: 3) motif containing peptide.

8. A method of diminishing bleeding in a subject in need thereof, the method comprising administering to the subject a therapeutically effective amount of the synthetic platelet composition of claim 1, and wherein the bleeding in the subject is diminished.

9. The method of claim 8, wherein the composition is administered to the subject intravenously.

10. A method of diminishing bleeding in a subject in need thereof, the method comprising administering to the subject a therapeutically effective amount of the synthetic platelet composition of claim 2, and wherein the bleeding in the subject is diminished.

11. The method of claim 10, wherein the composition is administered to the subject intravenously.

12. A method of diminishing bleeding in a subject in need thereof, the method comprising administering to the subject a therapeutically effective amount of the synthetic platelet composition of claim 3, and wherein the bleeding in the subject is diminished.

13. The method of claim 12, wherein the composition is administered to the subject intravenously.

14. A method of diminishing bleeding in a subject in need thereof, the method comprising administering to the subject a therapeutically effective amount of the synthetic platelet composition of claim 4, and wherein the bleeding in the subject is diminished.

15. The method of claim 14, wherein the composition is administered to the subject intravenously.

16. A method of diminishing bleeding in a subject in need thereof, the method comprising administering to the subject a therapeutically effective amount of the synthetic platelet composition of claim 5, and wherein the bleeding in the subject is diminished.

17. The method of claim 16, wherein the composition is administered to the subject intravenously.

18. A method of diminishing bleeding in a subject in need thereof, the method comprising administering to the subject a therapeutically effective amount of the synthetic platelet composition of claim 6, and wherein the bleeding in the subject is diminished.

19. The method of claim 18, wherein the composition is administered to the subject intravenously.

20. A method of diminishing bleeding in a subject in need thereof, the method comprising administering to the subject a therapeutically effective amount of the synthetic platelet composition of claim 7, and wherein the bleeding in the subject is diminished.

21. The method of claim 20, wherein the composition is administered to the subject intravenously.

22. A nanoparticle therapeutic agent delivery vehicle comprising a polyhydroxy acid polymer, wherein the polyhydroxy acid polymer is conjugated with at least one polyethylene glycol acrylate molecule.

23. The composition of claim 22, wherein the polyhydroxy acid polymer comprises at least one of the group selected from poly-lactic-co-glycolic acid and poly-L-lactic acid.

24. The composition of claim 22, wherein the polyhydroxy acid polymer comprises a poly(lactic-co-glycolic acid)-poly-L-lysine (PLGA-PLL) copolymer.

25. The composition of claim 22, wherein the polyethylene glycol acrylate molecule is at least one selected from the group consisting of PEG 200, PEG 1000, PEG 1500, PEG 4600 and PEG 10,000.

26. The composition of claim 22, wherein the polyethylene glycol acrylate molecule comprises the polyethylene glycol diacrylate.

27. The composition of claim 22, wherein the nanoparticle therapeutic agent delivery vehicle further comprises a pharmaceutically acceptable carrier.

28. A nanoparticle therapeutic agent delivery vehicle comprising a poly(lactic-co-glycolic acid)-poly-L-lysine (PLGA-PLL) copolymer, wherein the a poly(lactic-co-glycolic acid)-poly-L-lysine (PLGA-PLL) copolymer is conjugated with at least one PEG acrylate molecule, and wherein the nanoparticle encapsulates at least one therapeutic agent.

29. The nanoparticle therapeutic agent delivery vehicle of claim 5, wherein the at least one therapeutic agent is CNTF.

30. A method of treating a subject in need thereof, the method comprising administering to the subject a therapeutically effective amount of the nanoparticle therapeutic agent delivery vehicle of claim 22, wherein the nanoparticle therapeutic agent delivery vehicle encapsulates at least one therapeutic agent, and wherein the subject is treated.

31. The method of claim 30, wherein the nanoparticle therapeutic agent delivery vehicle is administered to the subject intravenously.

32. The method of claim 30, wherein the at least one therapeutic agent is CNTF.

33. A method of treating a subject in need thereof, the method comprising administering to the subject a therapeutically effective amount of the nanoparticle therapeutic agent delivery vehicle of claim 23, wherein the nanoparticle therapeutic agent delivery vehicle encapsulates at least one therapeutic agent, and wherein the subject is treated.

34. The method of claim 33, wherein the nanoparticle therapeutic agent delivery vehicle is administered to the subject intravenously.

35. The method of claim 33, wherein the at least one therapeutic agent is CNTF.

36. A method of treating a subject in need thereof, the method comprising administering to the subject a therapeutically effective amount of the nanoparticle therapeutic agent delivery vehicle of claim 24, wherein the nanoparticle therapeutic agent delivery vehicle encapsulates at least one therapeutic agent, and wherein the subject is treated.

37. The method of claim 36, wherein the nanoparticle therapeutic agent delivery vehicle is administered to the subject intravenously.

38. The method of claim 36, wherein the at least one therapeutic agent is CNTF.

39. A method of treating a subject in need thereof, the method comprising administering to the subject a therapeutically effective amount of the nanoparticle therapeutic agent delivery vehicle of claim 25, wherein the nanoparticle therapeutic agent delivery vehicle encapsulates at least one therapeutic agent, and wherein the subject is treated.

40. The method of claim 39, wherein the nanoparticle therapeutic agent delivery vehicle is administered to the subject intravenously.

41. The method of claim 39, wherein the at least one therapeutic agent is CNTF.

42. A method of treating a subject in need thereof, the method comprising administering to the subject a therapeutically effective amount of the nanoparticle therapeutic agent delivery vehicle of claim 26, wherein the nanoparticle therapeutic agent delivery vehicle encapsulates at least one therapeutic agent, and wherein the subject is treated.

43. The method of claim 42, wherein the nanoparticle therapeutic agent delivery vehicle is administered to the subject intravenously.

44. The method of claim 42, wherein the at least one therapeutic agent is CNTF.

45. A method of treating a subject in need thereof, the method comprising administering to the subject a therapeutically effective amount of the nanoparticle therapeutic agent delivery vehicle of claim 27, wherein the nanoparticle therapeutic agent delivery vehicle encapsulates at least one therapeutic agent, and wherein the subject is treated.

46. The method of claim 45, wherein the nanoparticle therapeutic agent delivery vehicle is administered to the subject intravenously.

47. The method of claim 45, wherein the at least one therapeutic agent is CNTF.

48. A method of treating a subject in need thereof, the method comprising administering to the subject a therapeutically effective amount of the nanoparticle therapeutic agent delivery vehicle of claim 28, wherein the nanoparticle therapeutic agent delivery vehicle encapsulates at least one therapeutic agent, and wherein the subject is treated.

49. The method of claim 48, wherein the nanoparticle therapeutic agent delivery vehicle is administered to the subject intravenously.

50. The method of claim 48, wherein the at least one therapeutic agent is CNTF.

51. A method of treating a subject in need thereof, the method comprising administering to the subject a therapeutically effective amount of the nanoparticle therapeutic agent delivery vehicle of claim 29, wherein the nanoparticle therapeutic agent delivery vehicle encapsulates at least one therapeutic agent, and wherein the subject is treated.

52. The method of claim 51, wherein the nanoparticle therapeutic agent delivery vehicle is administered to the subject intravenously.

53. The method of claim 51, wherein the at least one therapeutic agent is CNTF.


SEQUENCE LISTING (6 sequences)

SEQ ID NO: 1 - protein, 3 aa, Homo sapiens
RGD

SEQ ID NO: 2 - protein, 4 aa, Homo sapiens
RGDS

SEQ ID NO: 3 - protein, 5 aa, Homo sapiens
GRGDS

SEQ ID NO: 4 - protein, 6 aa, Homo sapiens
GRADSP

SEQ ID NO: 5 - protein, 6 aa, Homo sapiens
KQAGDV

SEQ ID NO: 6 - protein, 3 aa, Homo sapiens
KGD